SEQUENCE LISTING (33 sequences)

SEQ ID NO: 1 - RNA, 19 nt, Artificial Sequence
  Description of Artificial Sequence Synthetic oligonucleotide
ggaucuuauuucuucggag

SEQ ID NO: 2 - protein, 7 aa, Artificial Sequence
  Description of Artificial Sequence Synthetic peptide
CWKKKKC

SEQ ID NO: 3 - protein, 9 aa, Artificial Sequence
  Description of Artificial Sequence Synthetic peptide
CWKKKKKKC

SEQ ID NO: 4 - protein, 11 aa, Artificial Sequence
  Description of Artificial Sequence Synthetic peptide
CWKKKKKKKKC

SEQ ID NO: 5 - protein, 13 aa, Artificial Sequence
  Description of Artificial Sequence Synthetic peptide
CWKKKKKKKKKKC

SEQ ID NO: 6 - protein, 12 aa, Artificial Sequence
  Description of Artificial Sequence Synthetic peptide
CWKKKKCKKKKC

SEQ ID NO: 7 - protein, 12 aa, Artificial Sequence
  Description of Artificial Sequence Synthetic peptide
CWKKKKHKKKKC

SEQ ID NO: 8 - protein, 13 aa, Artificial Sequence
  Description of Artificial Sequence Synthetic peptide
CWHKKKKHKKKKC

SEQ ID NO: 9 - protein, 14 aa, Artificial Sequence
  Description of Artificial Sequence Synthetic peptide
CWHKKKKHKKKKHC

SEQ ID NO: 10 - protein, 9 aa, Artificial Sequence
  Description of Artificial Sequence Synthetic peptide
RRRRRRRRR

SEQ ID NO: 11 - protein, 18 aa, Artificial Sequence
  Description of Artificial Sequence Synthetic peptide
RRRRRRRRRRRRRRRRRR

SEQ ID NO: 12 - protein, 9 aa, Artificial Sequence
  Description of Artificial Sequence Synthetic peptide
KKKKKKKKK

SEQ ID NO: 13 - protein, 18 aa, Artificial Sequence
  Description of Artificial Sequence Synthetic peptide
KKKKKKKKKKKKKKKKKK

SEQ ID NO: 14 - protein, 20 aa, Artificial Sequence
  Description of Artificial Sequence Synthetic peptide
KKKKKKKKKKKKKKKKKKKK

SEQ ID NO: 15 - protein, 25 aa, Artificial Sequence
  Description of Artificial Sequence Synthetic peptide
KKKKKKKKKKKKKKKKKKKKKKKKK

SEQ ID NO: 16 - protein, 10 aa, Artificial Sequence
  Description of Artificial Sequence Synthetic peptide
KKKKKKKKKK

SEQ ID NO: 17 - protein, 15 aa, Artificial Sequence
  Description of Artificial Sequence Synthetic peptide
KKKKKKKKKKKKKKK

SEQ ID NO: 18 - protein, 25 aa, Artificial Sequence
  Description of Artificial Sequence Synthetic peptide
KKKKKKKKKKKKKKKKKKKKKKKKK

SEQ ID NO: 19 - protein, 15 aa, Artificial Sequence
  Description of Artificial Sequence Synthetic peptide
KKKKKKKKKKKKKKK

SEQ ID NO: 20 - protein, 30 aa, Artificial Sequence
  Description of Artificial Sequence Synthetic peptide
KKKKKKKKKKKKKKKKKKKKKKKKKKKKKK

SEQ ID NO: 21 - protein, 36 aa, Artificial Sequence
  Description of Artificial Sequence Synthetic peptide
KGSKKAVTKAQKKDGKKRKRSRKESYSVYVYKVLKQ

SEQ ID NO: 22 - protein, 18 aa, Artificial Sequence
  Description of Artificial Sequence Synthetic peptide
VRLPPPVRLPPPVRLPPP

SEQ ID NO: 23 - protein, 18 aa, Artificial Sequence
  Description of Artificial Sequence Synthetic peptide
PLKPLKPLKPLKPLKPLK

SEQ ID NO: 24 - protein, 18 aa, Artificial Sequence
  Description of Artificial Sequence Synthetic peptide
CWKKKKKKKKKKKKKKKC

SEQ ID NO: 25 - protein, 32 aa, Artificial Sequence
  Description of Artificial Sequence Synthetic peptide
CWKKKKKKKKKKKKKKCKKKKKKKKKKKKKKC

SEQ ID NO: 26 - protein, 32 aa, Artificial Sequence
  Description of Artificial Sequence Synthetic peptide
CWKKKKKKKKKKKKKKHKKKKKKKKKKKKKKC

SEQ ID NO: 27 - protein, 31 aa, Artificial Sequence
  Description of Artificial Sequence Synthetic peptide
CWHKKKKKKKKKKKKKHKKKKKKKKKKKKKC

SEQ ID NO: 28 - protein, 30 aa, Artificial Sequence
  Description of Artificial Sequence Synthetic peptide
CWHKKKKKKKKKKKKHKKKKKKKKKKKKHC

SEQ ID NO: 29 - protein, 5 aa, Artificial Sequence
  Description of Artificial Sequence Synthetic peptide
CWKKC

SEQ ID NO: 30 - protein, 8 aa, Artificial Sequence
  Description of Artificial Sequence Synthetic peptide
CWKKCKKC

SEQ ID NO: 31 - protein, 8 aa, Artificial Sequence
  Description of Artificial Sequence Synthetic peptide
CWKKHKKC

SEQ ID NO: 32 - protein, 9 aa, Artificial Sequence
  Description of Artificial Sequence Synthetic peptide
CWHKKHKKC

SEQ ID NO: 33 - protein, 10 aa, Artificial Sequence
  Description of Artificial Sequence Synthetic peptide
CWHKKHKKHC

(19) United States
(12) Patent Application Publication
(10) Pub. No.: US 2007/0269892 A1
(43) Pub. Date: Nov. 22, 2007

(54) FORMULATIONS FOR INTRACELLULAR DELIVERY dsRNA

(75) Inventors: Roger C. Adami, Snohomish, WA (US); Kevin G. Rice, Iowa City, IA (US)

Correspondence Address: NASTECH PHARMACEUTICAL COMPANY INC, 3830 MONTE VILLA PARKWAY, BOTHELL, WA, 98021-7266, US

(73) Assignees: NASTECH PHARMACEUTICAL COMPANY INC., Bothell, WA (US); UNIVERSITY OF IOWA RESEARCH FOUNDATION, Iowa City, IA (US)

(21) Appl. No.: 11/750,979

(22) Filed: May 18, 2007

Related U.S. Application Data
(60) Provisional application No. 60/747,622, filed on May 18, 2006.

(51) Int. Cl. C12N 15/09

(52) U.S. Cl. 435/455; 530/352

(57) ABSTRACT

What is described is a composition for delivery of a RNA molecule to a cell, comprising: a double stranded RNA (dsRNA) molecule of about 15 to about 40 base pairs; and a cationic polymer peptide comprising at least two cysteine residues and at least one tryptophan residues with one or more repeats consisting of between about five to about 20 positively charged residues, a method for producing such composition, a method of using such composition to deliver dsRNA to a cell, and a method of using such composition to inhibit expression of a target gene.

### CROSS REFERENCE TO RELATED APPLICATIONS

This patent application claims priority under 35 U.S.C. § 119(e) to U.S. Provisional Application No. 60/747,622 filed May 18, 2006, which application is incorporated herein by reference in its entirety.

### BACKGROUND OF THIS DISCLOSURE

Delivering nucleic acids into animal and plant cells to achieve a specific biological effect has long been an important objective of molecular biology research and development. Recent developments in the areas of gene therapy, antisense therapy and RNA interference (RNAi) therapy have created a need to develop more efficient means for introducing nucleic acids into cells.

A variety of methods are available for delivering nucleic acids artificially into cells. These include transfection via calcium phosphate, cationic lipid, and lipsomal delivery. Nucleic acids can also be introduced into cells by electroporation and viral transduction. However, there are several disadvantages to these methods including cytotoxicity, antigenicity, complement activation, solubility, blood compatibility and stability.

In an attempt to overcome the problems associated with the current methodologies used to deliver nucleic acids into cells, several low molecular weight (LMW) carrier peptides have been developed. These offer the advantage of controlled synthesis and defined purity, which then allows for a strategy of systematic optimization to increase expression levels and eliminate side effects. However, when tested for in vivo efficacy, LMW carriers have been shown to lack sufficient stability to remain intact during circulation and thereby do not significantly protect the nucleic acid from premature metabolism in tissue (Kwoh et al., 1999). Recently, certain cross-linking agents have been applied to form caged DNA condensates by template polymerization, but thus far, these have not been shown to be transfection competent (Trubetskoy et al, 1998; 1999). In seeking a solution to the relative instability of LMW carriers, increased stability should not be over-emphasized to the detriment of gene transfer efficiency and/or gene expression.

Thus, there remains a long-standing need in the art for better tools and methods to deliver nucleic acids, peptides and other pharmacological agents into cells, particularly in view of the fact that existing techniques for delivering cargo into cells are limited by poor efficiency and/or high toxicity of the delivery reagents. Related needs exist for improved methods and formulations to deliver an effective amount, in an active and enduring state, and using non-toxic delivery vehicles, to selected cells, tissues, or compartments to mediate regulation of gene expression in a manner that will alter a phenotype or disease state of the targeted cells.

### SUMMARY OF THIS DISCLOSURE

One aspect of this disclosure is a composition for delivery of an RNA molecule to a cell, comprising: a double stranded RNA (dsRNA) molecule of about 15 to about 40 base pairs; and a cationic polymer peptide comprising at least two cysteine residues and at least one tryptophan residues with one or more repeats consisting of between about five to about 20 positively charged residues. In one embodiment, the peptide comprises two or more cysteine residues and one or more tryptophan residues with one or more repeats consisting of positively charged residues. In another embodiment, the peptide comprises two or more cysteine residues, one or more tryptophan residues, two or more histidine residues with one or more repeats consisting of positively charged residues. In another embodiment, the peptide has an amino acid sequence comprising Cys-Trp-(Lys)_{X}-Cys (SEQ ID NO:29); Cys-Trp-(Lys)_{X}-Cys-(Lys)_{X}-Cys (SEQ ID NO:30); Cys-Trp-(Lys)_{X}-His-(Lys)_{X}-Cys (SEQ ID NO:31); Cys-Trp-His-(Lys)_{X}-His-(Lys)_{X}-Cys (SEQ ID NO:32) or Cys-Trp-His-(Lys)_{X}-His-(Lys)_{X}-His-Cys (SEQ ID NO:33), wherein X is greater than one and the total number of non-lysine residues is less than 18. In another embodiment, the peptide is pegylated. In another embodiment, the peptide is acetylated. In another embodiment, the composition is reacted with a dialdehyde to cross-link a multiplicity of peptides. In another embodiment, the peptides cross-link after binding to dsRNA by forming interpeptide disulfide bonds. In another embodiment, the peptide is derivatized with a single N-glycan.

Another aspect of this disclosure is a method of stabilizing a double stranded ribonucleic acid (dsRNA)-peptide condensate, comprising: preparing a dsRNA-peptide condensate by mixing a dsRNA with a peptide in water, and cross-linking at least a portion of the peptides within the condensate. In one embodiment, the method further comprises the steps of: selecting a peptide comprising two or more lysine residues, and cross-linking the peptides after binding to dsRNA by reacting with a dialdehyde. In another embodiment, the method further comprises the steps of: selecting a peptide comprising two or more cysteine residues, and cross-linking the peptides after binding to dsRNA by forming interpeptide disulfide bonds.

Another aspect of this disclosure is a method for delivering a double stranded (ds)RNA molecule to a cell, comprising: preparing a condensate comprising a dsRNA molecule of about 15 to about 40 base pairs; preparing a dsRNA-peptide condensate by mixing a dsRNA with a peptide in water, cross-linking at least a portion of the peptides within the condensate, and treating a cell with said condensate. In one embodiment, the method further comprises the steps of: selecting a peptide comprising two or more lysine residues, and cross-linking the peptides after binding to dsRNA by reacting with a dialdehyde. In another embodiment, the method further comprises the steps of: selecting a peptide comprising two or more cysteine residues, and cross-linking the peptides after binding to dsRNA by forming interpeptide disulfide bonds.

Another aspect of this disclosure is a method for inhibiting expression of a gene in a cell comprising: preparing a condensate comprising a dsRNA molecule of about 15 to about 40 base pairs; preparing a dsRNA-peptide condensate by mixing a dsRNA with a peptide in water, cross-linking at least a portion of the peptides within the condensate, and treating a cell with said condensate.

### BRIEF DESCRIPTION OF DRAWINGS

FIG. 1 shows results from an in vitro assay using 9L/LacZ cells, depicting uptake of condensed peptide-siRNA particles (samples designated NT-18 through NT-21) at two concentrations (10 nM and 100 nM) and N/P ratios (N/P=2 and N/P=10), and knockdown of target gene expression (i.e., Lac-Z). The solid bars represent target gene expression, clear bars represent total protein expression, and the solid line represents peptide-siRNA uptake (i.e., fluorescence intensity).

FIG. 2 shows results from an in vitro assay using 9L/LacZ cells, depicting uptake of condensed peptide-siRNA particles designated NT-18 through NT-25 formulated in the presence of pluronic F127 and, condensates formed with the peptides designated PN0593 and PN0594 and, knockdown of target gene expression (i.e., Lac-Z). The solid bars represent total protein expression, clear bars represent target gene (i.e., Lac-Z) expression, and the solid line represents uptake (i.e., fluorescence intensity).

### DETAILED DESCRIPTION OF THIS DISCLOSURE

The present disclosure satisfies these needs and fulfills additional objects and advantages by providing novel compositions and methods for the introduction of nucleic acids into cells. In particular, the present disclosure relates to compositions for the intracellular delivery of a short interfering nucleic acid (siNA), or a precursor thereof, comprising a peptide/siRNA condensate for efficient siRNA mediated reduction of a targeted RNA.

The peptide of a peptide/siRNA condensate of the present disclosure is a cationic polymer peptide comprising at least one, up to about 100 positively charged residues, for example, at least five, up to about 20 positively charged residues. The amino acid sequence of the peptide is represented as follows: NH₂—(X)_{N}-amide, where N is one to about 100, for example, wherein N is five to about 20, and wherein X is a positively charged residue (e.g., a Lys or a Arg residue). In certain embodiments, a cationic peptide disclosed herein may have up to about 30 positively charged residues. In another embodiment of this disclosure, the peptide comprises two or more cysteine residues and one or more tryptophan residues with one or more repeats consisting of positively charged residues. In yet another embodiment of this disclosure, the peptide comprises two or more cysteine residues, one or more tryptophan residues, two or more histidine residues with one or more repeats consisting of positively charged residues. Based on the above description, exemplary peptides of the present disclosure have the following amino acid sequence: Cys-Trp-(Lys)_{X}-Cys (SEQ ID NO:24); Cys-Trp-(Lys)_{X}-Cys-(Lys)_{X}-Cys (SEQ ID NO:25); Cys-Trp-(Lys)_{X}-His-(Lys)_{X}-Cys (SEQ ID NO:26); Cys-Trp-His-(Lys)_{X}-His-(Lys)_{X}-Cys (SEQ ID NO:27) or Cys-Trp-His-(Lys)_{X}-His-(Lys)_{X}-His-Cys (SEQ ID NO:28), where X is equal to or greater than one but does not exceed the value calculated by subtracting the total number of non-lysine residues from 18. In some embodiments exemplary peptides of the present disclosure have the following amino acid sequence: Cys-Trp-(Lys)_{X}-Cys (SEQ ID NO:29); Cys-Trp-(Lys)_{X}-Cys-(Lys)_{X}-Cys (SEQ ID NO:30); Cys-Trp-(Lys)_{X}-His-(Lys)_{X}-Cys (SEQ ID NO:31); Cys-Trp-His-(Lys)_{X}-His-(Lys)_{X}-Cys (SEQ ID NO:32) or Cys-Trp-His-(Lys)_{X}-His-(Lys)_{X}-His-Cys (SEQ ID NO:33), wherein X is greater than one, preferably in a range of from 1 to 100, and the total number of non-lysine residues is less than 18, including wherein the number of non-lysine residues is zero. In some embodiments, peptides disclosed herein may include blocking or cross-linkable end groups at the amino (i.e., NH₂) and/or carboxy (i.e., COOH) termini, for example a Ac modified amino termini, a amide modified carboxy termini, or both a Ac modified amino termini and a amide modified carboxy termini, a PEG2k-Mal modified amino termini along with an amide modified carboxy termini, a PEG10k-Mal modified amino termini along with a amide modified carboxy termini, or other cross-linkable amino and/or carboxy terminal modification.

In additional detailed embodiments, the peptide may be pegylated to improve stability and/or efficacy, particularly in the context of in vivo administration. In yet another embodiment, the peptide may be acetylated (i.e., Ac).

In another embodiment of this disclosure, unpaired amines are exploited to provide effectively cross-linked peptide/siRNA condensates. Increasing the stability of peptide/siRNA condensates is thus achieved by introducing dialdehyde groups, such as glutaraldehyde, to cross-link surface amine groups on the peptides. LMW peptides cross-linked in this manner condense siRNA into small condensates (which may also be referred to as particles) with improved stability, as demonstrated by increased resistance to shear stress induced fragmentation. Glutaraldehyde-crosslinked condensates are also significantly more resistant to in vitro metabolism by serum endonucleases.

In another embodiment, the cross-linking peptides of this disclosure are prepared by replacing lysine residues with cysteine residues to provide low molecular weight siRNA condensing peptides that spontaneously cross-link, after binding to siRNA, by forming interpeptide disulfide bonds. The peptides thus contain multiple sulfhydryl groups designed to spontaneously polymerize and cross-link when bound to siRNA. The stability of cross-linked peptide/siRNA condensates is dependent, at least in part, on the number of cysteines incorporated into the peptide. Disulfide bond formation in this manner decreases siRNA condensates particle size, relative to control peptide/siRNA condensates, and prevents dissociation of peptide siRNA condensates.

In yet another embodiment of the present disclosure, the LMW peptide/siRNA condensate exhibits metabolic stability and reversibility. The LMW peptide portions of the siRNA condensates incorporate multiple cysteine residues that allow the peptides to undergo oxidation to form interpeptide disulfide bonds while bound to siRNA. Once in a target cell, the disulfide cross-links are reduced, releasing siRNA for efficient reduction in gene expression levels. The reducing environment of the endosome is believed to mediate disulfide reduction and siRNA release.

The peptide/siRNA condensates of this disclosure provide their own multicomponent peptide condensed siRNA formulations and can be further combined with other gene therapy agents, such as matrices, carriers and targeting agents, for even more effective in vivo therapies.

The cross-linking peptides themselves may be covalently derivatized with, for example, polyethylene glycol (PEG). PEG-peptides form a steric layer on the surface of siRNA condensates, mask siRNA condensate recognition by the reticuloendothelial system and increase siRNA condensate solubility by blocking the formation of aggregates. In some embodiments herein, a PEG modification may be appended to an amino and/or a carboxyl termini of a cationic peptide. In some embodiments, appending of a PEG is mediated by a linking moiety such as a maleimido (Mal) or a disulfide (e.g., S—S bond) linkage.

In still further embodiments, the self-cross-linking peptides of this disclosure may be converted into cross-linking and targeting peptides by the addition of targeting units. For example, target specificity is achieved by derivatizing a cross-linking peptide with a single N-glycan resulting in glycopeptides that direct targeting to either the asialoglycoprotein receptor on hepatocytes or the mannose receptor on liver Kupffer cells.

siRNA co-condensates can thus be prepared using systematically determined admix ratios of cross-linking glycopeptide and PEG-peptide. The backbone of cross-linking peptides are chemically modifiable by reduction of the amide linkages to install secondary amines designed to buffer endosomes and allow siRNA condensates to release into the cytosol of target cells. Once in the cytosol, cross-linked siRNA condensates slowly release siRNA following, for example, disulfide reduction. Decreasing siRNA metabolism by increasing siRNA condensate stability prolongs the half-life of siRNA and produces a longer duration of targeted reduction in gene expression in vivo. The present disclosure thus overcomes various limitations of current gene delivery systems.

As provided herein, nucleic acid condensates are, for example, those comprising a nucleic acid and at least two low molecular weight peptides or peptide polymers with sufficient positive charge to bind to a nucleic acid, the peptides being linked via a glutaraldehyde crosslinker; and comprising a nucleic acid and an amount of glutaraldehyde-crosslinked nucleic acid-binding peptides that form a non-covalently linked peptide-nucleic acid condensate.

Stable nucleic acid condensates are provided, comprising nucleic acids and an amount of glutaraldehyde-crosslinked, nucleic acid-binding peptides effective to stabilize the nucleic acid. Nucleic acid condensates with in vivo stability comprise a nucleic acid and an amount of glutaraldehyde-crosslinked, nucleic acid-binding peptides effective to stabilize the nucleic acid under in vivo conditions.

Methods of stabilizing a nucleic acid-peptide condensate comprise cross-linking nucleic acid-binding peptides within the condensate with at least a first glutaraldehyde crosslinker; whereas methods of protecting a nucleic acid from degradation comprise preparing a nucleic acid-peptide condensate and cross-linking at least a portion of the peptides within the condensate using a glutaraldehyde crosslinker.

The self-crosslinking aspects of this disclosure provide a cationic linker comprising sufficient positive charge to bind to a nucleic acid and at least two thiol groups; a low molecular weight cationic linker comprising sufficient positive charge to bind to a nucleic acid and at least two thiol groups; and cationic linkers wherein the linker comprises a positively-charged peptide, a cationic polymer, or a cationic lipid with sufficient positively-charged amine groups to bind to a nucleic acid.

Nucleic acid condensing agents are provided comprising a low molecular weight cationic linker with sufficient positive charge to bind to a nucleic acid and sufficient thiol groups to form a self-crosslinked construct that induces a bound nucleic acid to condense.

Further provided are peptides comprising sufficient positively-charged residues to bind to a nucleic acid and capable of forming a disulfide-bonded peptide; and peptides comprising sufficient positively-charged residues to bind to a nucleic acid and at least two thiol groups.

The peptides (which may be used in the form of a polymer) are between about 3 and about 100; between 3 and about 50 amino acids in length; between about 4 and about 50 amino acids in length; between about 5 and about 50 amino acids in length; between about 10 and about 50 amino acids in length; between about 5 and about 40 amino acids in length; between about 5 and about 30 amino acids in length; between about 5 and about 20 amino acids in length; between about 5 and about 10 amino acids in length; between about 25 and about 30 amino acids in length; between about 20 and about 25 amino acids in length; between about 15 and about 20 amino acids in length; and between about 10 and about 15 amino acids in length.

The peptides are further about 3, 4, 5, 6, 7, 8, 9, 10, 11, 12, 13, 14, 15, 16, 17, 18, 19, 20, 21, 22, 23, 24, 25, 25, 27, 28, 29, 30, 31, 32, 33, 34, 35, 36, 37, 38, 39, 40, 41, 42, 43, 44, 45, 46, 47, 48, 49 or 50 or so amino acids in length.

The peptides may comprise between about 2 and about 45 positively-charged residues; between about 3 and about 45 positively-charged residues; between about 4 and about 45 positively-charged residues; between about 5 and about 45 positively-charged residues; between about 10 and about 45 positively-charged residues; between about 15 and about 45 positively-charged residues; between about 20 and about 45 positively-charged residues; between about 25 and about 45 positively-charged residues; between about 30 and about 45 positively-charged residues; between about 35 and about 45 positively-charged residues; between about 40 and about 45 positively-charged residues.

This can be achieved by comprising between about 2 and about 45 positively-charged lysine residues; between about 3 and about 45 positively-charged lysine residues; between about 4 and about 45 positively-charged lysine residues; between about 5 and about 45 positively-charged lysine residues; between about 10 and about 45 positively-charged lysine residues; between about 15 and about 45 positively-charged lysine residues; between about 20 and about 45 positively-charged lysine residues; between about 25 and about 45 positively-charged lysine residues; between about 30 and about 45 positively-charged lysine residues; between about 35 and about 45 positively-charged lysine residues; between about 40 and about 45 positively-charged lysine residues.

The peptides may be thiolylated substantially polylysine peptides. They may comprise at least 3, 4, 5, 6, 7, 8 or so thiol groups or may have only two thiol groups.

At least one, two, three, four, five, six, seven, eight or so cysteine residue may provide at least one of the thiol groups. Two cysteine residues are suitable examples. The peptides may be alkylated, wherein it may be that the at least a first cysteine residue is alkylated. D-amino acid residues may be employed if desired.

The peptides may be dispersed within a pharmaceutically acceptable medium, bound to a nucleic acid, associated with a matrix, associated with a carrier or a targeting ligand, covalently linked to a targeting ligand, covalently linked to at least a first glycosyl unit, thereby forming a glycopeptide targeting ligand, covalently linked to at least a first oligosaccharide unit to form a glycopeptide targeting ligand, or may be both bound to a nucleic acid and associated with a matrix, carrier or a targeting ligand.

The peptides may thus be summarized as being between about 3 and about 50 amino acids in length, comprising sufficient positively-charged residues to bind to a nucleic acid and at least two thiol groups, such as two cysteine residues. The peptides may comprise sufficient positively-charged residues to bind to a nucleic acid and a number of thiol groups sufficient to form a reversibly-linked nucleic acid-peptide composition that dissociates under endosomal conditions.

Nucleic-acid cross-linking peptides may comprise an amount of positively-charged residues effective to bind nucleic acid and at least two thiol groups effective to form spontaneous peptide-crosslinks sufficient to produce ionic-crosslinked nucleic acids upon contact, optionally with at least a first glycosyl unit.

Exemplary nucleic acid condensates are, for example, those comprising a nucleic acid and a nucleic acid-binding peptide that comprises a plurality of positively-charged residues and at least two thiol groups and those comprising a nucleic acid condensate that comprises a nucleic acid and a nucleic acid-binding peptide that comprises a plurality of positively-charged residues and at least two thiol groups.

Peptide-linked nucleic acid condensates may comprise nucleic acids and an amount of positively-charged, double-thiol-containing nucleic acid-binding peptides effective to form a non-covalently linked peptide-nucleic acid condensate; or nucleic acids and an amount of positively-charged, double-thiol-containing nucleic acid-binding peptides effective to form interpeptide disulfide bonds, thereby condensing nucleic acids in non-covalent contact with the disulfide-bonded peptides; or a nucleic acid and nucleic acid-binding peptides, wherein the peptides each comprise a plurality of positively-charged residues and at least two thiol groups and form a condensed nucleic acid particle of between about 10 nm and about 20 nm in diameter upon contact with nucleic acids.

Stable nucleic acid condensates are, for example, those comprising a nucleic acid and at least two positively-charged nucleic acid-binding peptides that comprise an amount of thiol groups effective to stabilize the nucleic acid; nucleic acid condensates with in vivo stability may comprise a nucleic acid and at least two positively-charged nucleic acid-binding peptides that comprise an amount of thiol groups effective to stabilize the nucleic acid under in vivo conditions.

Reversibly-bound nucleic acid-peptide condensates comprise nucleic acids and an amount of positively-charged nucleic acid-binding peptides with an amount of thiol groups effective to form a nucleic acid-peptide condensate that is substantially stable in an extracellular biological environment and that releases nucleic acids upon contact with an intracellular endosome.

Targeted siRNA delivery complexes comprise a targeting ligand and a nucleic acid condensate of nucleic acids (e.g., an siRNA) and positively-charged nucleic acid-binding peptides that comprise an amount of thiol groups effective to condense and stabilize the nucleic acids.

Multimolecular complexes of the present disclosure comprise a carrier, a targeting ligand and a nucleic acid condensate of nucleic acids (e.g., an siRNA) and positively-charged nucleic acid-binding peptides that comprise an amount of thiol groups effective to condense and stabilize the nucleic acids. The multimolecular complexes may further comprise a biocompatible matrix.

Nucleic acid condensates with a particle size of between about 10 nm and about 100 nm in diameter; between about 10 nm and about 50 nm in diameter; and between about 10 nm and about 20 nm in diameter are included, but are not limiting of this disclosure.

Preferred cationic linkers may be a first and a second low molecular weight peptide, preferably of between about 6 and about 20 amino acids in length or between about 6 and about 10 amino acids in length or between about 10 and about 20 amino acids in length.

The first and second peptides may each preferably comprise between about four and about eight. Lysine residues that mediate non-covalent binding of the peptides to the nucleic acid; and at least two, three or four thiol groups and wherein the peptides are cross-linked to each other by reaction of the thiol groups.

The purified low molecular weight synthetic peptides themselves form aspects of the present disclosure, wherein the peptide comprises sufficient positive charge to bind to a nucleic acid and sufficient thiol groups to form disulfide-crosslinked peptides that induce nucleic acids to condense upon contact with a population of the peptides.

A population of purified nucleic-acid condensing peptides is provided, wherein the peptides are synthetic peptides of between about 6 and about 20 amino acids in length, comprise an amount of positively-charged residues effective to bind nucleic acid, comprise at least two thiol groups effective to spontaneously cross-link peptides within the population and comprises an amount of secondary or tertiary amines effective to promote dissociation under endosomal conditions; wherein the population of peptides is effective to form a nucleic acid-peptide condensate that is substantially stable in an extracellular biological environment and that releases nucleic acids intracellularly in a manner effective to reduce gene expression of a target gene.

Co-condensates are particularly preferred, such as those comprising at least a first peptide operatively attached to at least a first stealthing agent and at least a second peptide operatively attached to at least a first targeting agent. Exemplary co-condensates are, for example, those comprising a population of peptides; wherein between about 5% and 20% of the peptides are operatively attached to PEG; between about 5% and 20% of the peptides are operatively attached to a glycosyl targeting unit; and the remainder of the peptides comprise about four Histidine or secondary or tertiary amine residues.

Kits of this disclosure comprising, in at least a first container: a plurality of low molecular weight peptides with sufficient positive charge to bind to a nucleic acid and an amount of glutaraldehyde effective to cross-link at least a portion of the peptides; or a plurality of low molecular weight peptides that each comprise at least two thiol groups and have sufficient positive charge to bind to nucleic acids, the peptides spontaneously forming intermolecular disulfide-crosslinks.

Methods of preparing a nucleic acid-peptide condensate comprise contacting a nucleic acid with at least two nucleic acid-binding peptides that have sufficient positive charge to bind to a nucleic acid; wherein: the nucleic acid-binding peptides are cross-linked with glutaraldehyde, thereby condensing the nucleic acid in non-covalent contact with the cross-linked peptides; or wherein the nucleic acid-binding peptides each comprise a thiol capacity sufficient to spontaneously form interpeptide cross-links, thereby condensing the nucleic acid in contact with the cross-linked peptides.

The cell may be located within an animal, wherein the nucleic acid condensate is administered to the animal.

Methods for providing a nucleic acid to an animal comprise providing to the animal an effective amount of a nucleic acid condensate that comprises the nucleic acid in functional association with a population of low molecular weight nucleic acid-binding peptides; wherein: the nucleic acid-binding peptides are cross-linked with glutaraldehyde in a manner effective to stabilize the nucleic acid under in vivo conditions; or wherein the nucleic acid-binding peptides each comprise at least two thiol groups and spontaneously form disulfide cross-links in a manner effective to stabilize the nucleic acid under in vivo conditions.

Various uses of one or more composition in accordance with the present disclosure include the manufacture of medicaments for treating a wide range of diseases and disorders amenable to modulation of endogenous gene expression are further encompassed.

### Definitions

As used herein, the term “low molecular weight” or “LMW” refers to a peptide having less than about 25 amino acid residues.

As used herein, the term “high molecular weight” or “HMW” refers to a peptide have greater than about 25 amino acid residues.

As used herein, the term “cross-linking” refers formation of intermolecular disulfide bonds or intermolecular reaction via reagents such as glutaraldehyde.

As used herein, the term “positively charged amino acid” refers to an amino acid having a positive charge at about pH 3 to pH 6

As used herein, the term “condensate” as it refers to the condensed matter phase of the peptide/siRNA is a particle or aggregate with a defined size; particle formation is characterized by the ability to be analyzed by light scattering and/or electron microscopy.

As used herein, the term “short interfering nucleic acid”, “siNA”, “short interfering RNA”, “siRNA”, “short interfering nucleic acid molecule”, “short interfering oligonucleotide molecule”, or “chemically-modified short interfering nucleic acid molecule”, refers to any nucleic acid molecule capable of directly or indirectly inhibiting or down regulating gene expression or viral replication, for example by mediating RNA interference “RNAi” or gene silencing in a sequence-specific manner. Within exemplary embodiments, the siNA is a double-stranded polynucleotide molecule comprising self-complementary sense and antisense regions, wherein the antisense region comprises a nucleotide sequence that is complementary to a nucleotide sequence in a target nucleic acid molecule for down regulating expression, or a portion thereof, and the sense region comprises a nucleotide sequence corresponding to (i.e., which is substantially identical in sequence to) the target nucleic acid sequence or portion thereof.

“siNA” means a small interfering nucleic acid, for example a siRNA, that is a short-length double-stranded nucleic acid (or optionally a longer precursor thereof), and which is not unacceptably toxic in target cells. The length of useful siNAs within this disclosure will in certain embodiments be optimized at a length of approximately 21 to 23 base pairs (bp) long. However, there is no particular limitation in the length of useful siNAs, including siRNAs. For example, siNAs can initially be presented to cells in a precursor form that is substantially different than a final or processed form of the siNA that will exist and exert gene silencing activity upon delivery, or after delivery, to the target cell. Precursor forms of siNAs may, for example, include precursor sequence elements that are processed, degraded, altered, or cleaved at or following the time of delivery to yield a siNA that is active within the cell to mediate gene silencing. Thus, in certain embodiments, useful siNAs within this disclosure will have a precursor length, for example, of approximately 100-200 base pairs, 50-100 base pairs, or less than about 50 base pairs, which will yield an active, processed siNA within the target cell. In other embodiments, a useful siNA or siNA precursor will be approximately 10 to 49 bp, 15 to 35 bp, or about 21 to 30 bp in length.

The term “gene” refers to a DNA sequence that comprises control and coding sequences necessary for the production of a polypeptide or precursor thereof. The polypeptide can be encoded by a full length coding sequence or by any portion of the coding sequence so long as the desired activity is retained.

As used herein the term “cell” is meant to include both prokaryotic (e.g., bacterial) and eukaryotic (e.g., mammalian or plant) cells. Cells may be of somatic or germ line origin, may be totipotent or pluripotent, and may be dividing or non-dividing. Cells can also be derived from or can comprise a gamete or an embryo, a stem cell, or a fully differentiated cell. Thus, the term “cell” is meant to retain its usual biological meaning and can be present in any organism such as, for example, a bird, a plant, and a mammal, including, for example, a human, a cow, a sheep, an ape, a monkey, a pig, a dog, and a cat. Within certain aspects, the term “cell” refers specifically to mammalian cells, such as human cells, that contain one or more siRNA molecule(s) of the present disclosure.

As used herein, the term “RNA” is meant to include polynucleotide molecules comprising at least one ribonucleotide residue. The term “ribonucleotide” is meant to include nucleotides with a hydroxyl group at the 2′ position of a β-D-ribo-furanose moiety. The term RNA includes, for example, double-stranded RNAs; single-stranded RNAs; and isolated RNAs such as partially purified RNA, essentially pure RNA, synthetic RNA, recombinantly produced RNA, as well as altered RNA that differ from naturally occurring RNA by the addition, deletion, substitution and/or alteration of one or more nucleotides. Such alterations can include addition of non-nucleotide material, such as to the end(s) of the siRNA or internally, for example at one or more nucleotides of the RNA. As disclosed in detail herein, nucleotides in the siRNA molecules of the instant disclosure can also comprise non-standard nucleotides, such as non-naturally occurring nucleotides or chemically synthesized nucleotides or deoxynucleotides. These altered RNAs can be referred to as analogs or analogs of naturally-occurring RNA.

“Antisense RNA” is an RNA strand having a sequence complementary to a target gene mRNA, and thought to induce RNAi by binding to the target gene mRNA. “Sense RNA” has a sequence complementary to the antisense RNA, and annealed to its complementary antisense RNA to form siRNA. These antisense and sense RNAs have been conventionally synthesized with an RNA synthesizer.

As used herein, the term “RNAi construct” is a generic term used throughout the specification to include small interfering RNAs (siRNAs), hairpin RNAs, and other RNA species which can be cleaved in vivo to form siRNAs. RNAi constructs herein also include expression vectors (also referred to as RNAi expression vectors (e.g., a plasmid expression vector)) capable of giving rise to transcripts which form dsRNAs or hairpin RNAs in cells, and/or transcripts which can produce siRNAs in vivo. Optionally, the siRNA include single strands or double strands of siRNA.

A siHybrid molecule is a double-stranded nucleic acid that has a similar function to siRNA. Instead of a double-stranded RNA molecule, an siHybrid is comprised of an RNA strand and a DNA strand. Preferably, the RNA strand is the antisense strand as that is the strand that binds to the target mRNA. The siHybrid created by the hybridization of the DNA and RNA strands have a hybridized complementary portion and preferably at least one 3′ overhanging end.

By “highly conserved sequence region” is meant, a nucleotide sequence of one or more regions in a target gene which does not vary significantly from one generation to the other or from one biological system to the other.

By “sense region” is meant a nucleotide sequence of a siNA molecule having complementarity to an antisense region of the siNA molecule. In addition, the sense region of a siNA molecule can comprise a nucleic acid sequence having homology with a target nucleic acid sequence.

By “antisense region” is meant a nucleotide sequence of a siNA molecule having complementarity to a target nucleic acid sequence. In addition, the antisense region of a siNA molecule can optionally comprise a nucleic acid sequence having complementarity to a sense region of the siNA molecule.

A pharmacological composition or formulation refers to a composition or formulation in a form suitable for administration, e.g., systemic administration, into a cell or patient, including for example a human. Suitable forms, in part, depend upon the use or the route of entry, for example oral, transdermal, or by injection. Such forms should not prevent the composition or formulation from reaching a target cell (i.e., a cell to which the negatively charged nucleic acid is desirable for delivery). For example, pharmacological compositions injected into the blood stream should be soluble. Other factors are known in the art, and include considerations such as toxicity.

By “systemic administration” is meant in vivo systemic absorption or accumulation of drugs in the blood stream followed by distribution throughout the entire body. Administration routes which lead to systemic absorption include, without limitation: intravenous, subcutaneous, intraperitoneal, inhalation, oral, intranasal, intrapulmonary and intramuscular. Each of these administration routes exposes the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant disclosure can potentially localize the drug, for example, in certain tissue types, such as the tissues of the reticular endothelial system (RES). A liposome formulation that can facilitate the association of drug with the surface of cells, such as, lymphocytes and macrophages is also useful. This approach may provide enhanced delivery of the drug to target cells by taking advantage of the specificity of macrophage and lymphocyte immune recognition of abnormal cells, such as cancer cells.

By “pharmaceutically acceptable formulation” is meant, a composition or formulation that allows for the effective distribution of the nucleic acid molecules of the instant disclosure in the physical location most suitable for their desired activity. Non-limiting examples of agents suitable for formulation with the nucleic acid molecules of the instant disclosure include: P-glycoprotein inhibitors (such as Pluronic P85), which can enhance entry of drugs into the CNS (Jolliet-Riant and Tillement, Fundam. Clin. Pharmacol. 13:16-26, 1999); biodegradable polymers, such as poly (DL-lactide-coglycolide) microspheres for sustained release delivery after intracerebral implantation (Emerich, D. F. et al., Cell Transplant 8:47-58, 1999, Alkermes, Inc., Cambridge, Mass.; and loaded nanoparticles, such as those made of polybutylcyanoacrylate, which can deliver drugs across the blood brain barrier and can alter neuronal uptake mechanisms (Prog. Neuropsychopharmacol Biol. Psychiatry 23:941-949, 1999). Other non-limiting examples of delivery strategies for the nucleic acid molecules of the instant disclosure include material described in Boado et al., J. Pharm. Sci. 87:1308-1315, 1998; Tyler et al., FEBS Lett. 421:280-284, 1999; Pardridge et al., PNAS USA. 92:5592-5596, 1995; Boado, Adv. Drug Delivery Rev. 15:73-107, 1995; Aldrian-Herrada et al., Nucleic Acids Res. 26:4910-4916, 1998; and Tyler et al., PNAS USA. 96:7053-7058, 1999.

A pharmaceutically effective dose is that dose required to prevent, modulate, inhibit the occurrence of, or treat (alleviate a symptom to some extent, preferably all of the symptoms) a disease state. The pharmaceutically effective dose depends on the type of disease, the composition used, the route of administration, the type of mammal being treated, the physical characteristics of the specific mammal under consideration, concurrent medication, and other factors that those skilled in the medical arts will recognize. Generally, an amount between about 0.1 mg/kg and about 100 mg/kg body weight/day of active ingredients is administered dependent upon potency of the negatively charged polymer.

A “non-nucleotide” means any group or compound that can be incorporated into a nucleic acid chain in the place of one or more nucleotide units, including either sugar and/or phosphate substitutions, and allows the remaining bases of, for example, an siRNA, to exhibit their enzymatic activity. The group or compound can be abasic in that it does not contain a commonly recognized nucleotide base, such as adenosine, guanine, cytosine, uracil or thymidine, for example at the C1 position of the sugar.

The term “ligand” refers to any compound or molecule, such as a drug, peptide, hormone, or neurotransmitter that is capable of interacting with another compound, such as a receptor, either directly or indirectly. The receptor that interacts with a ligand can be present on the surface of a cell or can alternately be an intracellular receptor. Interaction of the ligand with the receptor can result in a biochemical reaction, or can simply be a physical interaction or association.

By “asymmetric hairpin” as used herein is meant a linear siNA molecule comprising an antisense region, a loop portion that can comprise nucleotides or non-nucleotides, and a sense region that comprises fewer nucleotides than the antisense region to the extent that the sense region has enough complementary nucleotides to base pair with the antisense region and form a duplex with loop. For example, an asymmetric hairpin siNA molecule of this disclosure can comprise an antisense region having length sufficient to mediate RNAi in a T-cell (e.g., about 19 to about 22 (e.g., about 19, 20, 21, or 22) nucleotides) and a loop region comprising about 4 to about 8 (e.g., about 4, 5, 6, 7, or 8) nucleotides, and a sense region having about 3 to about 18 (e.g., about 3, 4, 5, 6, 7, 8, 9, 10, 11, 12, 13, 14, 15, 16, 17, or 18) nucleotides that are complementary to the antisense region. The asymmetric hairpin siNA molecule can also comprise a 5′-terminal phosphate group that can be chemically modified. The loop portion of the asymmetric hairpin siNA molecule can comprise nucleotides, non-nucleotides, linker molecules, or conjugate molecules as described herein.

By “asymmetric duplex” as used herein is meant a siNA molecule having two separate strands comprising a sense region and an antisense region, wherein the sense region comprises fewer nucleotides than the antisense region to the extent that the sense region has enough complementary nucleotides to base pair with the antisense region and form a duplex. For example, an asymmetric duplex siNA molecule of this disclosure can comprise an antisense region having length sufficient to mediate RNAi in a T-cell (e.g., about 19 to about 22 (e.g., about 19, 20, 21, or 22) nucleotides) and a sense region having about 3 to about 18 (e.g., about 3, 4, 5, 6, 7, 8, 9, 10, 11, 12, 13, 14, 15, 16, 17, or 18) nucleotides that are complementary to the antisense region.

By “modulate gene expression” is meant that the expression of a target gene is upregulated or downregulated, which can include upregulation or downregulation of mRNA levels present in a cell, or of mRNA translation, or of synthesis of protein or protein subunits, encoded by the target gene. Modulation of gene expression can be determined also be the presence, quantity, or activity of one or more proteins or protein subunits encoded by the target gene that is up regulated or down regulated, such that expression, level, or activity of the subject protein or subunit is greater than or less than that which is observed in the absence of the modulator (e.g., a siRNA). For example, the term “modulate” can mean “inhibit,” but the use of the word “modulate” is not limited to this definition. Modulation of gene expression may, for example, be either direct (e.g., directly targeting the target gene mRNA) or indirect (e.g., directed to a mRNA of a gene that regulates expression of the targeted gene (such as a transcription factor or kinase)).

By “inhibit”, “down-regulate”, “knockdown” or “reduce” expression, it is meant that the expression of the gene, or level of RNA molecules or equivalent RNA molecules encoding one or more proteins or protein subunits, or level or activity of one or more proteins or protein subunits encoded by a target gene, is reduced below that observed in the absence of the nucleic acid molecules (e.g., siNA) of this disclosure. In one embodiment, inhibition, down-regulation or reduction with an siNA molecule is below that level observed in the presence of an inactive or attenuated molecule. In another embodiment, inhibition, down-regulation, or reduction with siNA molecules is below that level observed in the presence of, for example, an siNA molecule with scrambled sequence or with mismatches. In another embodiment, inhibition, down-regulation, or reduction of gene expression with a nucleic acid molecule of the instant disclosure is greater in the presence of the nucleic acid molecule than in its absence.

Gene “silencing” refers to partial or complete loss-of-function through targeted inhibition of gene expression in a cell and may also be referred to as “knockdown”. Depending on the circumstances and the biological problem to be addressed, it may be preferable to partially reduce gene expression. Alternatively, it may be desirable to reduce gene expression as much as possible. The extent of silencing may be determined by methods known in the art, some of which are summarized in International Publication No. WO 99/32619. Depending on the assay, quantification of gene expression permits detection of various amounts of inhibition that may be desired in certain embodiments of this disclosure, including prophylactic and therapeutic methods, which will be capable of knocking down target gene expression, in terms of mRNA levels or protein levels or activity, for example, by equal to or greater than about 10%, 30%, 50%, 75% 90%, 95% or 99% of baseline (i.e., normal) or other control levels, including elevated expression levels as may be associated with particular disease states or other conditions targeted for therapy.

The phrase “inhibiting expression of a target gene” refers to the ability of a siNA of this disclosure to initiate gene silencing of the target gene. To examine the extent of gene silencing, samples or assays of the organism of interest or cells in culture expressing a particular construct are compared to control samples lacking expression of the construct. Control samples (lacking construct expression) are assigned a relative value of 100%. Inhibition of expression of a target gene is achieved when the test value relative to the control is about 90%, often 50%, and in certain embodiments 25-0%. Suitable assays include, e.g., examination of protein or mRNA levels using techniques known to those of skill in the art such as dot blots, northern blots, in situ hybridization, ELISA, immunoprecipitation, enzyme function, as well as phenotypic assays known to those of skill in the art.

By “target nucleic acid” or “nucleic acid target” or “target RNA” or “RNA target” or “target DNA” or “DNA target” is meant any nucleic acid sequence whose expression or activity is to be modulated. The target nucleic acid can be DNA or RNA and is not limited to single strand forms.

The term “biologically active molecule” as used herein, refers to compounds or molecules that are capable of eliciting or modifying a biological response in a system. Non-limiting examples of biologically active siNA molecules either alone or in combination with other molecules contemplated by the instant disclosure include therapeutically active molecules such as antibodies, cholesterol, hormones, antivirals, peptides, proteins, chemotherapeutics, small molecules, vitamins, co-factors, nucleosides, nucleotides, oligonucleotides, enzymatic nucleic acids, antisense nucleic acids, triplex forming oligonucleotides, 2,5-A chimeras, siNA, dsRNA, allozymes, aptamers, decoys and analogs thereof. Biologically active molecules of this disclosure also include molecules capable of modulating the pharmacokinetics and/or pharmacodynamics of other biologically active molecules, for example, lipids and polymers such as polyamines, polyamides, polyethylene glycol and other polyethers.

By “cap structure” is meant chemical modifications, which have been incorporated at either terminus of the oligonucleotide (see, for example, Adamic et al., U.S. Pat. No. 5,998,203, incorporated by reference herein). These terminal modifications protect the nucleic acid molecule from exonuclease degradation, and may help in delivery and/or localization within a cell. The cap may be present at the 5′-terminus (5′-cap) or at the 3′-terminal (3′-cap) or may be present on both termini. In non-limiting examples, the 5′-cap includes, but is not limited to, glyceryl, inverted deoxy abasic residue (moiety); 4′,5′-methylene nucleotide; 1-(beta-D-erythrofuranosyl) nucleotide, 4′-thio nucleotide; carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety.

Non-limiting examples of the 3′-cap include, but are not limited to, glyceryl, inverted deoxy abasic residue (moiety), 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Lyer, 1993, Tetrahedron 49, 1925; incorporated by reference herein).

By “detectable level of cleavage” is meant cleavage of target RNA (and formation of cleaved product RNAs) to an extent sufficient to discern cleavage products above the background of RNAs produced by random degradation of the target RNA. Production of cleavage products from 1-5% of the target RNA is sufficient to detect above the background for most methods of detection.

By “biological system” is meant, material, in a purified or unpurified form, from biological sources, including but not limited to human, animal, plant, insect, bacterial, viral or other sources, wherein the system comprises the components required for RNAi activity. The term “biological system” includes, for example, a cell, tissue, or organism, or extract thereof. The term biological system also includes reconstituted RNAi systems that can be used in an in vitro setting.

The term “biodegradable linker” as used herein, refers to a nucleic acid or non-nucleic acid linker molecule that is designed as a biodegradable linker to connect one molecule to another molecule, for example, a biologically active molecule to a siNA molecule of this disclosure or the sense and antisense strands of a siNA molecule of this disclosure. The biodegradable linker is designed such that its stability can be modulated for a particular purpose, such as delivery to a particular tissue or cell type. The stability of a nucleic acid-based biodegradable linker molecule can be modulated by using various chemistries, for example combinations of ribonucleotides, deoxyribonucleotides, and chemically-modified nucleotides, such as 2′-O-methyl, 2′-fluoro, 2′-amino, 2′-O-amino, 2′-C-allyl, 2′-O-allyl, and other 2′-modified or base modified nucleotides. The biodegradable nucleic acid linker molecule can be a dimer, trimer, tetramer or longer nucleic acid molecule, for example, an oligonucleotide of about 2, 3, 4, 5, 6, 7, 8, 9, 10, 11, 12, 13, 14, 15, 16, 17, 18, 19, or 20 nucleotides in length, or can comprise a single nucleotide with a phosphorus-based linkage, for example, a phosphoramidate or phosphodiester linkage. The biodegradable nucleic acid linker molecule can also comprise nucleic acid backbone, nucleic acid sugar, or nucleic acid base modifications.

By “abasic” is meant sugar moieties lacking a base or having other chemical groups in place of a base at the 1′ position, see for example Adamic et al., U.S. Pat. No. 5,998,203.

By “unmodified nucleoside” is meant one of the bases adenine, cytosine, guanine, thymine, or uracil joined to the 1′ carbon of beta.-D-ribo-furanose.

By “modified nucleoside” is meant any nucleotide base which contains a modification in the chemical structure of an unmodified nucleotide base, sugar and/or phosphate.

In connection with 2′-modified nucleotides as described for the present disclosure, by “amino” is meant 2′-NH₂ or 2′-O—NH₂, which can be modified or unmodified. Such modified groups are described, for example, in Eckstein et al., U.S. Pat. No. 5,672,695 and Matulic-Adamic et al., U.S. Pat. No. 6,248,878.

“Inverted repeat” refers to a nucleic acid sequence comprising a sense and an antisense element positioned so that they are able to form a double stranded siRNA when the repeat is transcribed. The inverted repeat may optionally include a linker or a heterologous sequence such as a self-cleaving ribozyme between the two elements of the repeat. The elements of the inverted repeat have a length sufficient to form a double stranded RNA. Typically, each element of the inverted repeat is about 15 to about 100 nucleotides in length, preferably about 20-30 base nucleotides, preferably about 20-25 nucleotides in length, e.g., 20, 21, 22, 23, 24, 25, 26, 27, 28, 29, or 30 nucleotides in length.

“Large double-stranded RNA” refers to any double-stranded RNA having a size greater than about 40 bp for example, larger than 100 bp or more particularly larger than 300 bp. The sequence of a large dsRNA may represent a segment of an mRNA or the entire mRNA. The maximum size of the large dsRNA is not limited herein. The double-stranded RNA may include modified bases where the modification may be to the phosphate sugar backbone or to the nucleoside. Such modifications may include a nitrogen or sulfur heteroatom or any other modification known in the art.

The double-stranded structure may be formed by self-complementary RNA strand such as occurs for a hairpin or a micro RNA or by annealing of two distinct complementary RNA strands.

“Overlapping” refers to when two RNA fragments have sequences which overlap by a plurality of nucleotides on one strand, for example, where the plurality of nucleotides (nt) numbers as few as 2-5 nucleotides or by 5-10 nucleotides or more.

“One or more dsRNAs” refers to dsRNAs that differ from each other on the basis of sequence.

By “complementarity” is meant that a nucleic acid can form hydrogen bond(s) with another nucleic acid sequence by either traditional Watson-Crick or other non-traditional types. In reference to the nucleic molecules of the present disclosure, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al., CSH Symp. Quant. Biol. LII, 1987, pp. 123-133; Frier et al., Proc. Nat. Acad. Sci. USA 83:9373-9377, 1986; Turner et al., J. Am. Chem. Soc. 109:3783-3785, 1987. A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, or 10 nucleotides out of a total of 10 nucleotides in the first oligonucleotide being based paired to a second nucleic acid sequence having 10 nucleotides represents 50%, 60%, 70%, 80%, 90%, and 100% complementary respectively). “Perfectly complementary” means that all the contiguous residues of a nucleic acid sequence will hydrogen bond with the same number of contiguous residues in a second nucleic acid sequence.

By “subject” is meant an organism, tissue, or cell, which may include an organism as the subject or as a donor or recipient of explanted cells or the cells that are themselves a subject for siNA delivery. “Subject” therefore may refer to an organism, organ, tissue, or cell, including in vitro or ex vivo organ, tissue or cellular subjects, to which the nucleic acid molecules of this disclosure can be administered and enhanced by polypeptides described herein. Exemplary subjects include mammalian individuals or cells, for example human patients or cells.

By “comprising” is meant including, but not limited to, whatever follows the word “comprising.” Thus, use of the term “comprising” indicates that the listed elements are required or mandatory, but that other elements are optional and may or may not be present. By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in this disclosure for the listed elements. Thus, the phrase “consisting essentially of” indicates that the listed elements are required or mandatory, but that other elements are optional and may or may not be present depending upon whether or not they affect the activity or action of the listed elements.

In this specification and the appended claims, the singular forms of “a”, “an” and “the” include plural reference unless the context clearly dictates otherwise. The use of the alternative (e.g., “or”) should be understood to mean either one, both or any combination thereof of the alternatives.

As used herein, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, “about” or “consisting essentially of” mean±20% of the indicated range, value, or structure, unless otherwise indicated. As used herein, the terms “include” and “comprise” are used synonymously.

In addition, it should be understood that the individual compounds, or groups of compounds, derived from the various combinations of the structures and substituents described herein, are disclosed by the present application to the same extent as if each compound or group of compounds was set forth individually. Thus, selection of particular structures or particular substituents is within the scope of the present disclosure.

“Analog” or “analogue” as used herein refers to a chemical compound that is structurally similar to a parent compound (e.g., a peptide or protein), but differs slightly in composition (e.g., one atom or functional group is different, added, or removed). The analog may or may not have different chemical or physical properties than the original compound and may or may not have improved biological or chemical activity. For example, the analog may be more hydrophilic or it may have altered activity as compared to a parent compound. The analog may mimic the chemical or biological activity of the parent compound (i.e., it may have similar or identical activity), or, in some cases, may have increased or decreased activity. The analog may be a naturally or non-naturally occurring (e.g., chemically-modified, synthetic or recombinant) variant of the original compound. An example of an analog is a mutein (i.e., a protein analogue in which at least one amino acid is deleted, added, or substituted with another amino acid). Other types of analogs include isomers (enantiomers, diastereomers, and the like) and other types of chiral variants of a compound, as well as structural isomers.

“Derivative” or “derivatized” as used herein refers to a chemically or biologically modified version of a chemical compound (including an analog) that is structurally similar to a parent compound and (actually or theoretically) derivable from that parent compound. Generally, a “derivative” differs from an “analog” in that a parent compound may be the starting material to generate a “derivative,” whereas the parent compound may not necessarily be used as the starting material to generate an “analog.”

### Glutaraldehyed Cross-Linking

The stability of a siRNA formulation is fundamental to its successful application in vivo since metabolism results in the generation of fragmented siRNA that no longer mediates targeted reduction of gene expression. The present disclosure provides siRNA formulations with increased stability without increased toxicity. Basically, high affinity binding is providing using LMW carriers.

One aspect of this disclosure provides temporary stability through molecular cross-linking of carriers on siRNA condensates using aldehyde groups. Glutaraldehyde is a five carbon dialdehyde that has been used as a reagent to increase the tensile strength of transplanted pig heart valves and to develop controlled release microspheres for drug delivery (Jayakrishnan and Jameela, 1996; Jones et al., 1989; Gupta and Hung, 1989).

The chemical cross-linking of albumin lysines with glutaraldehyde leads to particles with controlled drug release properties (Lin et al., 1994; Royer and Lee, 1983). The degree of cross-linking directly affects the particle size, biodegradation, and release properties of drugs encapsulated in glutaraldehyde cross-linked microspheres (Jones et al., 1989; Gupta and Hung, 1989; Leong et al., 1998). Glutaraldehyde has not previously been connected with nucleic acid stability or delivery.

In the present disclosure, glutaraldehyde is used to cross-link the peptide/protein component of a siRNA condensate to improve their metabolic stability. This cross-linking enhances the stability of LMW peptide/siRNA condensates and leads to enhanced targeted reduction of gene expression.

As an example, the surface amine groups of a siRNA condensate were cross-linked using glutaraldehyde. Also, although not previously connected with nucleic stability or delivery, glutaraldehyde has a good safety record when used in a variety of prostheses (Gratzer et al., 1996) and has been used in diverse biomedical fields, particularly for developing cross-linked albumin microspheres for parenteral applications (Royer and Lee, 1983).

Glutaraldehyde cross-linking may increase the siRNA condensate stability resulting in prolonged funcationality of siRNA mediated reduction of gene expression.

### Small-Interfering Nucleic Acids (siNAs) and the RISC Complex

In mammalian cells, dsRNAs longer than 30 base pairs can activate the dsRNA-dependent kinase PKR and 2′-5′-oligoadenylate synthetase, normally induced by interferon. The activated PKR inhibits general translation by phosphorylation of the translation factor eukaryotic initiation factor 2α (eIF2α), while 2′-5′-oligoadenylate synthetase causes nonspecific mRNA degradation via activation of RNase L. By virtue of their small size (referring particularly to non-precursor forms), usually less than 30 base pairs, and most commonly between about 17-19, 19-21, or 21-23 base pairs, the siNAs of the present disclosure avoid activation of the interferon response.

In contrast to the nonspecific effect of long dsRNA, siRNA can mediate selective gene silencing in the mammalian system. Hairpin RNAs, with a short loop and 19 to 27 base pairs in the stem, also selectively silence expression of genes that are homologous to the sequence in the double-stranded stem. Mammalian cells can convert short hairpin RNA into siRNA to mediate selective gene silencing.

RISC mediates cleavage of single stranded RNA having sequence complementary to the antisense strand of the siRNA duplex. Cleavage of the target RNA takes place in the middle of the region complementary to the antisense strand of the siRNA duplex. Studies have shown that 21 nucleotide siRNA duplexes are most active when containing two nucleotide 3′-overhangs. Furthermore, complete substitution of one or both siRNA strands with 2′-deoxy (2′-H) or 2′-O-methyl nucleotides abolishes RNAi activity, whereas substitution of the 3′-terminal siRNA overhang nucleotides with deoxy nucleotides (2′-H) has been reported to be tolerated.

Studies have shown that replacing the 3′-overhanging segments of a 21-mer siRNA duplex having 2 nucleotide 3′ overhangs with deoxyribonucleotides does not have an adverse effect on RNAi activity. Replacing up to 4 nucleotides on each end of the siRNA with deoxyribonucleotides has been reported to be well tolerated whereas complete substitution with deoxyribonucleotides results in no RNAi activity.

Alternatively, the siNAs can be delivered as single or multiple transcription products expressed by a polynucleotide vector (e.g., a plasmid) encoding the single or multiple siNAs and directing their expression within target cells. In these embodiments the double-stranded portion of a final transcription product of the siRNAs to be expressed within the target cell can be, for example, 15 to 49 bp, 15 to 35 bp, or about 21 to 30 bp long. Within exemplary embodiments, double-stranded portions of siNAs, in which two strands pair up, are not limited to completely paired nucleotide segments, and may contain nonpairing portions due to mismatch (the corresponding nucleotides are not complementary), bulge (lacking in the corresponding complementary nucleotide on one strand), overhang, and the like. Nonpairing portions can be contained to the extent that they do not interfere with siNA formation. In more detailed embodiments, a “bulge” may comprise 1 to 2 nonpairing nucleotides, and the double-stranded region of siNAs in which two strands pair up may contain from about 1 to 7, or about 1 to 5 bulges. In addition, “mismatch” portions contained in the double-stranded region of siNAs may be present in numbers from about 1 to 7, or about 1 to 5. Most often in the case of mismatches, one of the nucleotides is guanine, and the other is uracil. Such mismatching may be attributable, for example, to a mutation from C to T, G to A, or mixtures thereof, in a corresponding DNA coding for sense RNA, but other causes are also contemplated. Furthermore, in the present disclosure the double-stranded region of siNAs in which two strands pair up may contain both bulge and mismatched portions in the approximate numerical ranges specified.

The terminal structure of siNAs of this disclosure may be either blunt or cohesive (overhanging) as long as the siNA retains its activity to silence or otherwise modulate expression of target genes. The cohesive (overhanging) end structure is not limited only to the 3′ overhang as reported by others. On the contrary, the 5′ overhanging structure may be included as long as it is capable of inducing a gene silencing effect such as by RNAi. In addition, the number of overhanging nucleotides is not limited to reported limits of 2 or 3 nucleotides, but can be any number as long as the overhang does not impair gene silencing activity of the siNA. For example, overhangs may comprise from about 1 to 8 nucleotides, more often from about 2 to 4 nucleotides. The total length of siNAs having cohesive end structure is expressed as the sum of the length of the paired double-stranded portion and that of a pair comprising overhanging single-strands at both ends. For example, in the exemplary case of a 19 bp double-stranded RNA with 4 nucleotide overhangs at both ends, the total length is expressed as 23 bp. Furthermore, since the overhanging sequence may have low specificity to a target gene, it is not necessarily complementary (antisense) or identical (sense) to the target gene sequence. Furthermore, as long as the siNA is able to maintain its gene silencing effect on the target gene, it may contain low molecular weight structure (for example a natural RNA molecule such as tRNA, rRNA or viral RNA, or an artificial RNA molecule), for example, in the overhanging portion at one end.

In addition, the terminal structure of the siNAs may have a stem-loop structure in which ends of one side of the double-stranded nucleic acid are connected by a linker nucleic acid, e.g., a linker RNA. The length of the double-stranded region (stem-loop portion) can be, for example, 15 to 49 bp, often 15 to 35 bp, and more commonly about 21 to 30 bp long. Alternatively, the length of the double-stranded region that is a final transcription product of siNAs to be expressed in a target cell may be, for example, approximately 15 to 49 bp, 15 to 35 bp, or about 21 to 30 bp long. When linker segments are employed, there is no particular limitation in the length of the linker as long as it does not hinder pairing of the stem portion. For example, for stable pairing of the stem portion and suppression of recombination between DNAs coding for this portion, the linker portion may have a clover-leaf tRNA structure. Even if the linker has a length that would hinder pairing of the stem portion, it is possible, for example, to construct the linker portion to include introns so that the introns are excised during processing of a precursor RNA into mature RNA, thereby allowing pairing of the stem portion. In the case of a stem-loop siRNA, either end (head or tail) of RNA with no loop structure may have a low molecular weight RNA. As described above, these low molecular weight RNAs may include a natural RNA molecule, such as tRNA, rRNA or viral RNA, or an artificial RNA molecule.

The siNA can also comprise a single stranded polynucleotide having nucleotide sequence complementary to nucleotide sequence in a target nucleic acid molecule or a portion thereof (for example, where such siNA molecule does not require the presence within the siNA molecule of nucleotide sequence corresponding to the target nucleic acid sequence or a portion thereof), wherein the single stranded polynucleotide can further comprise a terminal phosphate group, such as a 5′-phosphate (see for example, Martinez et al., Cell 110:563-574, 2002, and Schwarz et al., Molecular Cell 10:537-568, 2002, or 5′,3′-diphosphate.

As used herein, the term siNA molecule is not limited to molecules containing only naturally-occurring RNA or DNA, but also encompasses chemically-modified nucleotides and non-nucleotides. In certain embodiments, the short interfering nucleic acid molecules of this disclosure lack 2′-hydroxy (2′-OH) containing nucleotides. In certain embodiments short interfering nucleic acids do not require the presence of nucleotides having a 2′-hydroxy group for mediating RNAi and as such, short interfering nucleic acid molecules of this disclosure optionally do not include any ribonucleotides (e.g., nucleotides having a 2′-OH group). Such siNA molecules that do not require the presence of ribonucleotides within the siNA molecule to support RNAi can however have an attached linker or linkers or other attached or associated groups, moieties, or chains containing one or more nucleotides with 2′-OH groups. Optionally, siNA molecules can comprise ribonucleotides at about 5, 10, 20, 30, 40, or 50% of the nucleotide positions.

As used herein, the term siNA is meant to be equivalent to other terms used to describe nucleic acid molecules that are capable of mediating sequence specific RNAi, for example short interfering RNA (siRNA), double-stranded RNA (dsRNA), micro-RNA (mRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), and others.

In other embodiments, siNA molecules for use within this disclosure may comprise separate sense and antisense sequences or regions, wherein the sense and antisense regions are covalently linked by nucleotide or non-nucleotide linker molecules, or are alternately non-covalently linked by ionic interactions, hydrogen bonding, Van der Waals interactions, hydrophobic interactions, and/or stacking interactions.

siNAs for use within this disclosure can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure, for example wherein the double stranded region is about 19 base pairs). The antisense strand may comprise a nucleotide sequence that is complementary to a nucleotide sequence in a target nucleic acid molecule or a portion thereof, and the sense strand may comprise a nucleotide sequence corresponding to the target nucleic acid sequence or a portion thereof. Alternatively, the siNA can be assembled from a single oligonucleotide, where the self-complementary sense and antisense regions of the siNA are linked by means of a nucleic acid-based or non-nucleic acid-based linker(s).

Within additional embodiments, siNAs for intracellular delivery according to the methods and compositions of this disclosure can be a polynucleotide with a duplex, asymmetric duplex, hairpin or asymmetric hairpin secondary structure, having self-complementary sense and antisense regions, wherein the antisense region comprises a nucleotide sequence that is complementary to a nucleotide sequence in a separate target nucleic acid molecule or a portion thereof, and the sense region comprises a nucleotide sequence corresponding to the target nucleic acid sequence or a portion thereof.

Non-limiting examples of chemical modifications that can be made in an siNA include without limitation phosphorothioate internucleotide linkages, 2′-deoxyribonucleotides, 2′-O-methyl ribonucleotides, 2′-deoxy-2′-fluoro ribonucleotides, “universal base” nucleotides, “acyclic” nucleotides, 5-C-methyl nucleotides, and terminal glyceryl and/or inverted deoxy abasic residue incorporation. These chemical modifications, when used in various siNA constructs, are shown to preserve RNAi activity in cells while at the same time, dramatically increasing the serum stability of these compounds.

In a non-limiting example, the introduction of chemically-modified nucleotides into nucleic acid molecules provides a powerful tool in overcoming potential limitations of in vivo stability and bioavailability inherent to native RNA molecules that are delivered exogenously. For example, the use of chemically-modified nucleic acid molecules can enable a lower dose of a particular nucleic acid molecule for a given therapeutic effect since chemically-modified nucleic acid molecules tend to have a longer half-life in serum. Furthermore, certain chemical modifications can improve the bioavailability of nucleic acid molecules by targeting particular cells or tissues and/or improving cellular uptake of the nucleic acid molecule. Therefore, even if the activity of a chemically-modified nucleic acid molecule is reduced as compared to a native nucleic acid molecule, for example, when compared to an all-RNA nucleic acid molecule, the overall activity of the modified nucleic acid molecule can be greater than that of the native molecule due to improved stability and/or delivery of the molecule. Unlike native unmodified siNA, chemically-modified siNA can also minimize the possibility of activating interferon activity in humans.

The siNA molecules described herein, the antisense region of a siNA molecule of this disclosure can comprise a phosphorothioate internucleotide linkage at the 3′-end of said antisense region. In any of the embodiments of siNA molecules described herein, the antisense region can comprise about one to about five phosphorothioate internucleotide linkages at the 5′-end of said antisense region. In any of the embodiments of siNA molecules described herein, the 3′-terminal nucleotide overhangs of a siNA molecule of this disclosure can comprise ribonucleotides or deoxyribonucleotides that are chemically-modified at a nucleic acid sugar, base, or backbone. In any of the embodiments of siNA molecules described herein, the 3′-terminal nucleotide overhangs can comprise one or more universal base ribonucleotides. In any of the embodiments of siNA molecules described herein, the 3′-terminal nucleotide overhangs can comprise one or more acyclic nucleotides.

For example, in a non-limiting example, this disclosure features a chemically-modified short interfering nucleic acid (siNA) having about 1, 2, 3, 4, 5, 6, 7, 8 or more phosphorothioate internucleotide linkages in one siNA strand. In yet another embodiment, this disclosure features a chemically-modified short interfering nucleic acid (siNA) individually having about 1, 2, 3, 4, 5, 6, 7, 8 or more phosphorothioate internucleotide linkages in both siNA strands. The phosphorothioate internucleotide linkages can be present in one or both oligonucleotide strands of the siNA duplex, for example in the sense strand, the antisense strand, or both strands. The siNA molecules of this disclosure can comprise one or more phosphorothioate internucleotide linkages at the 3′-end, the 5′-end, or both of the 3′- and 5′-ends of the sense strand, the antisense strand, or both strands. For example, an exemplary siNA molecule of this disclosure can comprise about 1 to about 5 or more (e.g., about 1, 2, 3, 4, 5, or more) consecutive phosphorothioate internucleotide linkages at the 5′-end of the sense strand, the antisense strand, or both strands. In another non-limiting example, an exemplary siNA molecule of this disclosure can comprise one or more (e.g., about 1, 2, 3, 4, 5, 6, 7, 8, 9, 10, or more) pyrimidine phosphorothioate internucleotide linkages in the sense strand, the antisense strand, or both strands. In yet another non-limiting example, an exemplary siNA molecule of this disclosure can comprise one or more (e.g., about 1, 2, 3, 4, 5, 6, 7, 8, 9, 10, or more) purine phosphorothioate internucleotide linkages in the sense strand, the antisense strand, or both strands.

An siNA molecule may be comprised of a circular nucleic acid molecule, wherein the siNA is about 38 to about 70 (e.g., about 38, 40, 45, 50, 55, 60, 65, or 70) nucleotides in length having about 18 to about 23 (e.g., about 18, 19, 20, 21, 22, or 23) base pairs wherein the circular oligonucleotide forms a dumbbell shaped structure having about 19 base pairs and 2 loops.

A circular siNA molecule contains two loop motifs, wherein one or both loop portions of the siNA molecule is biodegradable. For example, a circular siNA molecule of this disclosure is designed such that degradation of the loop portions of the siNA molecule in vivo can generate a double-stranded siNA molecule with 3′-terminal overhangs, such as 3′-terminal nucleotide overhangs comprising about 2 nucleotides.

Modified nucleotides present in siNA molecules, preferably in the antisense strand of the siNA molecules, but also optionally in the sense and/or both antisense and sense strands, comprise modified nucleotides having properties or characteristics similar to naturally occurring ribonucleotides. For example, this disclosure features siNA molecules including modified nucleotides having a Northern conformation (e.g., Northern pseudorotation cycle, see for example Saenger, Principles of Nucleic Acid Structure, Springer-Verlag ed., 1984). As such, chemically modified nucleotides present in the siNA molecules of this disclosure, preferably in the antisense strand of the siNA molecules of this disclosure, but also optionally in the sense and/or both antisense and sense strands, are resistant to nuclease degradation while at the same time maintaining the capacity to mediate RNAi. Non-limiting examples of nucleotides having a northern configuration include locked nucleic acid (LNA) nucleotides (e.g., 2′-O, 4′-C-methylene-(D-ribofuranosyl) nucleotides); 2′-methoxyethoxy (MOE) nucleotides; 2′-methyl-thio-ethyl, 2′-deoxy-2′-fluoro nucleotides. 2′-deoxy-2′-chloro nucleotides, 2′-azido nucleotides, ribothymidine nucleotides and 2′-O-methyl nucleotides.

The sense strand of a double stranded siNA molecule may have a terminal cap moiety such as an inverted deoxybasic moiety, at the 3′-end, 5′-end, or both 3′ and 5′-ends of the sense strand.

Non-limiting examples of conjugates include conjugates and ligands described in Vargeese et al., U.S. application Ser. No. 10/427,160, filed Apr. 30, 2003, incorporated by reference herein in its entirety, including the drawings. In another embodiment, the conjugate is covalently attached to the chemically-modified siNA molecule via a biodegradable linker. In one embodiment, the conjugate molecule is attached at the 3′-end of either the sense strand, the antisense strand, or both strands of the chemically-modified siNA molecule. In another embodiment, the conjugate molecule is attached at the 5′-end of either the sense strand, the antisense strand, or both strands of the chemically-modified siNA molecule. In yet another embodiment, the conjugate molecule is attached both the 3′-end and 5′-end of either the sense strand, the antisense strand, or both strands of the chemically-modified siNA molecule, or any combination thereof. In one embodiment, a conjugate molecule of this disclosure comprises a molecule that facilitates delivery of a chemically-modified siNA molecule into a biological system, such as a cell. In another embodiment, the conjugate molecule attached to the chemically-modified siNA molecule is a poly ethylene glycol, human serum albumin, or a ligand for a cellular receptor that can mediate cellular uptake. Examples of specific conjugate molecules contemplated by the instant disclosure that can be attached to chemically-modified siNA molecules are described in Vargeese et al., U.S. Patent Application Publication No. 20030130186, published Jul. 10, 2003, and U.S. Patent Application Publication No. 20040110296, published Jun. 10, 2004. The type of conjugates used and the extent of conjugation of siNA molecules of this disclosure can be evaluated for improved pharmacokinetic profiles, bioavailability, and/or stability of siNA constructs while at the same time maintaining the ability of the siNA to mediate RNAi activity. As such, one skilled in the art can screen siNA constructs that are modified with various conjugates to determine whether the siNA conjugate complex possesses improved properties while maintaining the ability to mediate RNAi, for example in animal models as are generally known in the art.

A siNA may be further comprised of a nucleotide, non-nucleotide, or mixed nucleotide/non-nucleotide linker that joins the sense region of the siNA to the antisense region of the siNA. In one embodiment, a nucleotide linker can be a linker of >2 nucleotides in length, for example about 3, 4, 5, 6, 7, 8, 9, or 10 nucleotides in length. In another embodiment, the nucleotide linker can be a nucleic acid aptamer. By “aptamer” or “nucleic acid aptamer” as used herein is meant a nucleic acid molecule that binds specifically to a target molecule wherein the nucleic acid molecule has sequence that comprises a sequence recognized by the target molecule in its natural setting. Alternately, an aptamer can be a nucleic acid molecule that binds to a target molecule where the target molecule does not naturally bind to a nucleic acid. The target molecule can be any molecule of interest. For example, the aptamer can be used to bind to a ligand-binding domain of a protein, thereby preventing interaction of the naturally occurring ligand with the protein. This is a non-limiting example and those in the art will recognize that other embodiments can be readily generated using techniques generally known in the art. See, for example, Gold et al, Annu. Rev. Biochem. 64:763, 1995; Brody and Gold, J. Biotechnol. 74:5, 2000; Sun, Curr. Opin. Mol. Ther. 2:100, 2000; Kusser, J. Biotechnol. 74:27, 2000; Hermann and Patel, Science 287:820, 2000; and Jayasena, Clinical Chemistry 45:1628, 1999.

A non-nucleotide linker may be comprised of an abasic nucleotide, polyether, polyamine, polyamide, peptide, carbohydrate, lipid, polyhydrocarbon, or other polymeric compounds (e.g., polyethylene glycols such as those having between 2 and 100 ethylene glycol units). Specific examples include those described by Seela and Kaiser, Nucleic Acids Res. 18:6353, 1990, and Nucleic Acids Res. 15:3113, 1987; Cload and Schepartz, J. Am. Chem. Soc. 113:6324, 1991; Richardson and Schepartz, J. Am. Chem. Soc. 113:5109, 1991; Ma et al., Nucleic Acids Res. 21:2585, 1993, and Biochemistry 32:1751, 1993; Durand et al., Nucleic Acids Res. 18:6353, 1990; McCurdy et al., Nucleosides & Nucleotides 10:287, 1991; Jschke et al., Tetrahedron Lett. 34:301, 1993; Ono et al., Biochemistry 30:9914, 1991; Arnold et al., International Publication No. WO 89/02439; Usman et al., International Publication No. WO 95/06731; Dudycz et al., International Publication No. WO 95/11910 and Ferentz and Verdine, J. Am Chem. Soc. 113:4000, 1991.

The synthesis of a siNA molecule of this disclosure, which can be chemically-modified, comprises: (a) synthesis of two complementary strands of the siNA molecule; (b) annealing the two complementary strands together under conditions suitable to obtain a double-stranded siNA molecule. In another embodiment, synthesis of the two complementary strands of the siNA molecule is by solid phase oligonucleotide synthesis. In yet another embodiment, synthesis of the two complementary strands of the siNA molecule is by solid phase tandem oligonucleotide synthesis.

Oligonucleotides (e.g., certain modified oligonucleotides or portions of oligonucleotides lacking ribonucleotides) are synthesized using protocols known in the art, for example as described in Caruthers et al., Methods in Enzymology 211:3-19, 1992, Thompson et al., International PCT Publication No. WO 99/54459; Wincott et al., Nucleic Acids Res. 23:2677-2684, 1995; Wincott et al., Methods Mol. Bio. 74:59, 1997; Brennan et al., Biotechnol Bioeng. 61:33-45, 1998; and Brennan, U.S. Pat. No. 6,001,311. Synthesis of RNA, including certain siNA molecules of this disclosure, follows general procedures as described, for example, in Usman et al., J. Am. Chem. Soc. 109:7845, 1987; Scaringe et al., Nucleic Acids Res. 18:5433, 1990; and Wincott et al., Nucleic Acids Res. 23:2677-2684, 1995; Wincott et al., Methods Mol. Bio. 74:59, 1997.

The siNAs can be modified extensively to enhance stability by modification with nuclease resistant groups, for example, 2′-amino, 2′-C-allyl, 2′-fluoro, 2′-O-methyl, 2′-H. For a review see Usman and Cedergren, TIBS 17:34, 1992; Usman et al., Nucleic Acids Symp. Ser. 31:163, 1994. SiNA constructs can be purified by gel electrophoresis using general methods or can be purified by high pressure liquid chromatography and re-suspended in water.

Chemically synthesizing nucleic acid molecules with modifications (base, sugar and/or phosphate) can prevent their degradation by serum ribonucleases, which can increase their potency. See e.g., Eckstein et al., International Publication No. WO 92/07065; Perrault et al., Nature 344:565, 1990; Pieken et al., Science 253:314, 1991; Usman and Cedergren, Trends in Biochem. Sci. 17:334, 1992; Usman et al., International Publication No. WO 93/15187; and Rossi et al., International Publication No. WO 91/03162; Sproat, U.S. Pat. No. 5,334,711; Gold et al., U.S. Pat. No. 6,300,074. All of the above references describe various chemical modifications that can be made to the base, phosphate and/or sugar moieties of the nucleic acid molecules described herein.

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-fluoro, 2′-O-methyl, 2′-O-allyl, 2′-H, nucleotide base modifications. For a review see Usman and Cedergren, TIBS 17:34, 1992; Usman et al., Nucleic Acids Symp. Ser. 31:163, 1994; Burgin et al., Biochemistry 35:14090, 1996. Sugar modification of nucleic acid molecules have been extensively described in the art. See Eckstein et al., International Publication PCT No. WO 92/07065; Perrault et al., Nature 344:565-568, 1990; Pieken et al., Science 253:314-317, 1991; Usman and Cedergren, Trends in Biochem. Sci. 17:334-339, 1992; Usman et al. International Publication PCT No. WO 93/15187; Sproat, U.S. Pat. No. 5,334,711 and Beigelman et al., J. Biol. Chem. 270:25702, 1995; Beigelman et al., International PCT Publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., Karpeisky et al., Tetrahedron Lett. 39:1131, 1998; Earnshaw and Gait, Biopolymers (Nucleic Acid Sciences) 48:39-55, 1998; Verma and Eckstein, Annu. Rev. Biochem. 67:99-134, 1998; and Burlina et al., Bioorg. Med. Chem. 5:1999-2010, 1997. Such publications describe general methods and strategies to determine the location of incorporation of sugar, base and/or phosphate modifications and the like into nucleic acid molecules without modulating catalysis. In view of such teachings, similar modifications can be used as described herein to modify the siNA nucleic acid molecules of the instant disclosure so long as the ability of siNA to promote RNAi in cells is not significantly inhibited.

While chemical modification of oligonucleotide internucleotide linkages with phosphorothioate, phosphorodithioate, and/or 5′-methylphosphonate linkages improves stability, excessive modifications can cause some toxicity or decreased activity. Therefore, when designing nucleic acid molecules, the amount of these internucleotide linkages should be minimized. The reduction in the concentration of these linkages should lower toxicity, resulting in increased efficacy and higher specificity of these molecules.

In one embodiment, this disclosure features modified siNA molecules, with phosphate backbone modifications comprising one or more phosphorothioate, phosphorodithioate, methylphosphonate, phosphotriester, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications, see Hunziker and Leumann, Nucleic Acid Analogues: Synthesis and Properties, in Modern Synthetic Methods, VCH:331-417, 1995, and Mesmaeker et al., “Novel Backbone Replacements for Oligonucleotides, in Carbohydrate Modifications in Antisense Research,” ACS, 1994, pp. 24-39.

### Supplemental or Complementary Methods of Delivery

Supplemental or complementary methods for delivery of nucleic acid molecules for use within this disclosure are described, for example, in Akhtar et al., Trends Cell Bio. 2:139, 1992; “Delivery Strategies for Antisense Oligonucleotide Therapeutics,” ed. Akhtar, 1995, Maurer et al., Mol. Membr. Biol. 16:129-140, 1999; Hofland and Huang, Handb. Exp. Pharmacol. 137:165-192, 1999; and Lee et al., ACS Symp. Ser. 752:184-192, 2000. Sullivan et al., International PCT Publication No. WO 94/02595, further describes general methods for delivery of enzymatic nucleic acid molecules. These protocols can be utilized to supplement or complement delivery of virtually any nucleic acid molecule contemplated within this disclosure.

Nucleic acid molecules and polypeptides can be administered to cells by a variety of methods known to those of skill in the art, including, but not restricted to, administration within formulations that comprise the siNA and polypeptide alone, or that further comprise one or more additional components, such as a pharmaceutically acceptable carrier, diluent, excipient, adjuvant, emulsifier, buffer, stabilizer, preservative, and the like. In certain embodiments, the siNA and/or the polypeptide can be encapsulated in liposomes, administered by iontophoresis, or incorporated into other vehicles, such as hydrogels, cyclodextrins, biodegradable nanocapsules, bioadhesive microspheres, or proteinaceous vectors (see e.g., O'Hare and Normand, International PCT Publication No. WO 00/53722). Alternatively, a nucleic acid/peptide/vehicle combination can be locally delivered by direct injection or by use of an infusion pump. Direct injection of the nucleic acid molecules of this disclosure, whether subcutaneous, intramuscular, or intradermal, can take place using standard needle and syringe methodologies, or by needle-free technologies such as those described in Conry et al., Clin. Cancer Res. 5:2330-2337, 1999, and Barry et al., International PCT Publication No. WO 99/31262.

The compositions of the instant disclosure can be effectively employed as pharmaceutical agents. Pharmaceutical agents prevent, modulate the occurrence or severity of, or treat (alleviate one or more symptom(s) to a detectable or measurable extent) of a disease state or other adverse condition in a patient.

Thus within additional embodiments this disclosure provides pharmaceutical compositions and methods featuring the presence or administration of one or more polynucleic acid(s), typically one or more siNAs, combined, complexed, or conjugated with a polypeptide, optionally formulated with a pharmaceutically-acceptable carrier, such as a diluent, stabilizer, buffer, and the like.

The present disclosure satisfies additional objects and advantages by providing short interfering nucleic acid (siNA) molecules that modulate expression of genes associated with a particular disease state or other adverse condition in a subject. Typically, the siNA will target a gene that is expressed at an elevated level as a causal or contributing factor associated with the subject disease state or adverse condition. In this context, the siNA will effectively downregulate expression of the gene to levels that prevent, alleviate, or reduce the severity or recurrence of one or more associated disease symptoms. Alternatively, for various distinct disease models where expression of the target gene is not necessarily elevated as a consequence or sequel of disease or other adverse condition, down regulation of the target gene will nonetheless result in a therapeutic result by lowering gene expression (i.e., to reduce levels of a selected mRNA and/or protein product of the target gene). Alternatively, siNAs of this disclosure may be targeted to lower expression of one gene, which can result in upregulation of a “downstream” gene whose expression is negatively regulated by a product or activity of the target gene. Likewise, a siRNA of this disclosure may be targeted in a manner that will lower expression of one gene which subsequently down regulates expression of a downstream gene or activity that is positively regulated by a product or activity of the targeted gene (e.g., a kinase, a transcription factor or an adaptor molecule).

Further, a siNAs of the present disclosure may be administered in any form, for example transdermally or by local injection. Comparable methods and compositions are provided that target expression of one or more different genes associated with a selected disease condition in animal subjects, including any of a large number of genes whose expression is known to be aberrantly increased as a causal or contributing factor associated with the selected disease condition.

Negatively charged polynucleotides of this disclosure (e.g., RNA or DNA) can be administered to a patient by any standard means, with or without stabilizers, buffers, and the like, to form a pharmaceutical composition. When it is desired to use a liposome delivery mechanism, standard protocols for formation of liposomes can be followed. The compositions of the present disclosure may also be formulated and used as tablets, capsules or elixirs for oral administration, suppositories for rectal administration, sterile solutions, suspensions for injectable administration, and the other compositions known in the art.

The present disclosure also includes pharmaceutically acceptable formulations of the compositions described herein. These formulations include salts of the above compounds, e.g., acid addition salts, for example, salts of hydrochloric, hydrobromic, acetic acid, and benzene sulfonic acid.

The siNAs can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Methods for the delivery of nucleic acid molecules are described in Akhtar et al., Trends Cell Bio. 2:139, 1992; “Delivery Strategies for Antisense Oligonucleotide Therapeutics,” ed. Akhtar, 1995, Maurer et al., Mol. Membr. Biol. 16:129-140, 1999; Hofland and Huang, Handb. Exp. Pharmacol. 137:165-192, 1999; and Lee et al., ACS Symp. Ser. 752:184-192, 2000. Beigelman et al., U.S. Pat. No. 6,395,713 and Sullivan et al., PCT WO 94/02595 further describe the general methods for delivery of nucleic acid molecules. These protocols can be utilized for the delivery of virtually any nucleic acid molecule. Nucleic acid molecules can be administered to cells by a variety of methods known to those of skill in the art, including, but not restricted to, encapsulation in liposomes, by iontophoresis, or by incorporation into other vehicles, such as biodegradable polymers, hydrogels, cyclodextrins (see for example, Gonzalez et al., Bioconjugate Chem. 10:1068-1074, 1999; Wang et al., International PCT Publication Nos. WO 03/47518 and WO 03/46185), poly(lactic-co-glycolic acid) (PLGA) and PLCA microspheres (see for example U.S. Pat. No. 6,447,796 and U.S. Patent Application Publication No. US 2002130430), biodegradable nanocapsules, and bioadhesive microspheres, or by proteinaceous vectors (O'Hare and Normand, International PCT Publication No. WO 00/53722). Alternatively, the nucleic acid/vehicle combination is locally delivered by direct injection or by use of an infusion pump. Direct injection of the nucleic acid molecules of this disclosure, whether subcutaneous, intramuscular, or intradermal, can take place using standard needle and syringe methodologies, or by needle-free technologies such as those described in Conry et al., Clin. Cancer Res. 5:2330-2337, 1999, and Barry et al., International PCT Publication No. WO 99/31262. The molecules of the instant disclosure can be used as pharmaceutical agents. Pharmaceutical agents prevent, modulate the occurrence of, or treat (alleviate a symptom to some extent, preferably all of the symptoms) a disease state in a subject.

### Pharmaceutical Formulations

The present disclosure also includes pharmaceutically acceptable formulations of the compositions described herein. These formulations include salts of the above compounds, e.g., acid addition salts, for example, salts of hydrochloric, hydrobromic, acetic acid, and benzene sulfonic acid.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of this disclosure can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono-or diglycerides. In addition, fatty acids such as oleic acid find use in the preparation of injectables.

### EXAMPLES

The above generally describes the present disclosure, which is further exemplified by the following examples. These examples are described solely for purposes of illustration, and are not intended to limit the scope of this disclosure. Although specific terms and values have been employed herein, such terms and values will likewise be understood as exemplary and non-limiting to the scope of this disclosure.

### Example 1

### Materials and Methods Used

The present example illustrates the materials and methods used to assess the efficacy of the peptide/siRNA formulations of the present disclosure to enhance siRNA cell-uptake and permit siRNA mediated target gene knockdown activities. The cell culture conditions and protocols for each assay are explained below in detail. The protocols described below are for demonstrative purposes only and may be changed and/or modified accordingly.

siRNA Preparation

The nucleotide sequence of the exemplary siRNA (G1498) of the present disclosure is as follows: 5′-ggaucuuauuucuucggag-3′(SEQ ID NO:1). G1498 siRNA is suspended in Hyclone nuclease free water at a concentration of 5 mg/mL based on dry weight. The actual siRNA stock concentration is measured by making a 1 to 2000 and a 1 to 100 dilution of stock in water and measuring A260 on an Eppendorf UV Spectrophotometer. Quantification of concentration is made by calculating a ratio of the absorbance of these dilutions to an absorbance at 260 nm of 1.000 for 38.5 μg/mL of siRNA. This concentrated stock solution is stored in single-use aliquots at −80° C. and diluted to 20 μg/mL (2× concentration) when assayed. This will yield a final concentration of siRNA of 10 μg/mL, which is within the linear range of the SYBR gold binding standard. A 1 mL excess of siRNA solution will be made so that a multichannel pipette can be used.

### Cell Cultures

Example cell types that may be used include mouse tail fibroblast (MTF) and/or 9L/LacZ cells. MTF cells were derived from the tails of C57BL/6J mice. Tails were removed, immersed in 70% ethanol and then cut into small sections with a razor blade. The sections were washed three times with PBS and then incubated in a shaker at 37° C. with 0.5 mg/mL collagenase, 100 units/mL penicillin and 100 μg/mL streptomycin to disrupt tissue. Tail sections were then cultured in complete media (Dulbecco's Modified Essential Medium with 20% FBS, 1 mM sodium pyruvate, nonessential amino acids and 100 units/mL penicillin and 100 μg/mL streptomycin) until cells were established. Cells were cultured at 37° C., 5% CO₂ in complete media as outlined above.

The 9L/LacZ cell line constitutively expresses LacZ. 9L/LacZ cells were propagated in Dulbecco's modified Eagle's medium (DMEM) with 4.5 g/L glucose, 1 mM sodium pyruvate 90% and 10% fetal bovine serum at 37° C., 5% CO₂.

### Transfection

Cells are plated per well in 24-well plates the day before transfection in complete media. The siRNA is labeled, for example with Cy-5 conjugate. The peptide/siRNA condensates are added to cells previously washed with phosphate buffered saline (PBS). Cells were transfected for three hours at 37° C., 5% CO2. Cells were washed with PBS, treated with trypsin, and then analyzed by flow cytometry.

siRNA cell-uptake was measured by the intensity of intracellular Cy5 fluorescence. The cell-uptake assay is used to determine the percentage of cells that contain the Cy5-conjugated siRNA. Mean Fluorescence Intensity (MFI) is used to determine the relative mean quantity of Cy5-conjugated siRNA that entered the cells.

### LacZ Assay

The LacZ assay was performed three days post-transfection. Cells were rinsed with phosphate buffers saline (PBS) after removal of cell media. The PBS was aspirated and the cells were lysed by the addition of 50 μl M-PER™ to each well and incubating the cells for 15 minutes while gently shaking. Following cell lysis, 10 μl of cell lysate was removed from each well to perform a total protein assay (micro BCA kit, Pierce). In order to measure LacZ activity, 30 μl of cell lysate was transferred to a new plate and 30 μl of ALL-IN-ONE™ beta-galactosidase (β-gal) was added to each well of the new plate. The new plate was covered to avoid light exposure and incubated for 30 minutes at 37° C. Following the 30 minute incubation, the absorbance was read at 405 nM with a μQuant plate reader (Bio-Trek Instrument) to measure LacZ activity.

### Gold Dye Displacement Assay

Peptides (see Table 4, Example 2) are suspended in Hyclone nuclease free water. Test dilutions of this peptide stock are made in sterile, Nuclease-free eppendorf tubes at a 2× final concentration and buffered to pH 7.4 with 20 mM Phosphate. Nine concentrations of each peptide were made, corresponding to charge ratios of approximately 0.05 to 10. The molar ratios and μg/mL and μM concentrations of each peptide corresponding to these charge ratios are detailed in Tables 1, 2 and 3.

TABLE 1
Peptide:siRNA Molar Ratios and Corresponding Charge Ratio

| Charge Ratio | | | | | | | | | | | |
|---|---|---|---|---|---|---|---|---|---|---|---|
| (Peptide:siRNA) | PN861-2 | PN859-2 | PN860-2 | PN858-1 | PN924-1 | PN923-1 | PN933-2 | PN930-2 | PN925-2 | PN931-2 | PN907-2 |
| 0.04 | 0.17 | 0.09 | 0.17 | 0.09 | 0.08 | 0.06 | 0.16 | 0.10 | 0.06 | 0.10 | 0.05 |
| 0.08 | 0.35 | 0.17 | 0.35 | 0.17 | 0.16 | 0.13 | 0.31 | 0.21 | 0.13 | 0.21 | 0.10 |
| 0.16 | 0.69 | 0.35 | 0.69 | 0.35 | 0.31 | 0.25 | 0.63 | 0.42 | 0.25 | 0.42 | 0.21 |
| 0.31 | 1.39 | 0.69 | 1.39 | 0.69 | 0.63 | 0.50 | 1.25 | 0.83 | 0.50 | 0.83 | 0.42 |
| 0.63 | 2.78 | 1.39 | 2.78 | 1.39 | 1.25 | 1.00 | 2.50 | 1.67 | 1.00 | 1.67 | 0.83 |
| 1.25 | 5.56 | 2.78 | 5.56 | 2.78 | 2.50 | 2.00 | 5.00 | 3.33 | 2.00 | 3.33 | 1.67 |
| 2.50 | 11.1 | 5.6 | 11.1 | 5.6 | 5.0 | 4.0 | 10.0 | 6.7 | 4.0 | 6.7 | 3.3 |
| 5.00 | 22.2 | 11.1 | 22.2 | 11.1 | 10.0 | 8.0 | 20.0 | 13.3 | 8.0 | 13.3 | 6.7 |
| 10.0 | 44.4 | 22.2 | 44.4 | 22.2 | 20.0 | 16.0 | 40.0 | 26.7 | 16.0 | 26.7 | 13.3 |

TABLE 2
Peptide Molar Concentration and Corresponding Charge Ratio with 10 μg/mL siRNA

| Charge Ratio | | | | | | | | | | | |
|---|---|---|---|---|---|---|---|---|---|---|---|
| (Peptide:siRNA) | PN861-2 | PN859-2 | PN860-2 | PN858-1 | PN924-1 | PN923-1 | PN933-2 | PN930-2 | PN925-2 | PN931-2 | PN907-2 |
| 0.04 | 0.12 | 0.06 | 0.12 | 0.06 | 0.06 | 0.04 | 0.11 | 0.07 | 0.04 | 0.07 | 0.04 |
| 0.08 | 0.25 | 0.12 | 0.25 | 0.12 | 0.11 | 0.09 | 0.22 | 0.15 | 0.09 | 0.15 | 0.07 |
| 0.16 | 0.49 | 0.25 | 0.49 | 0.25 | 0.22 | 0.18 | 0.44 | 0.29 | 0.18 | 0.29 | 0.15 |
| 0.31 | 0.98 | 0.49 | 0.98 | 0.49 | 0.44 | 0.35 | 0.88 | 0.59 | 0.35 | 0.59 | 0.29 |
| 0.63 | 1.96 | 0.98 | 1.96 | 0.98 | 0.88 | 0.71 | 1.76 | 1.18 | 0.71 | 1.18 | 0.59 |
| 1.25 | 3.92 | 1.96 | 3.92 | 1.96 | 1.76 | 1.41 | 3.53 | 2.35 | 1.41 | 2.35 | 1.18 |
| 2.50 | 7.8 | 3.9 | 7.8 | 3.9 | 3.5 | 2.8 | 7.1 | 4.7 | 2.8 | 4.7 | 2.4 |
| 5.00 | 15.7 | 7.8 | 15.7 | 7.8 | 7.1 | 5.6 | 14.1 | 9.4 | 5.6 | 9.4 | 4.7 |
| 10.0 | 31.4 | 15.7 | 31.4 | 15.7 | 14.1 | 11.3 | 28.2 | 18.8 | 11.3 | 18.8 | 9.4 |

TABLE 3
Peptide Concentrations (μg/mL) and Corresponding Charge Ratios with 10 μg/mL siRNA

| Charge Ratio | | | | | | | | | | | |
|---|---|---|---|---|---|---|---|---|---|---|---|
| (Peptide:siRNA) | PN861-2 | PN859-2 | PN860-2 | PN858-1 | PN924-1 | PN923-1 | PN933-2 | PN930-2 | PN925-2 | PN931-2 | PN907-2 |
| 0.04 | 0.18 | 0.18 | 0.15 | 0.14 | 0.14 | 0.14 | 0.36 | 0.29 | 0.23 | 0.88 | 0.51 |
| 0.08 | 0.36 | 0.35 | 0.30 | 0.29 | 0.28 | 0.28 | 0.73 | 0.58 | 0.46 | 1.76 | 1.02 |
| 0.16 | 0.72 | 0.70 | 0.59 | 0.58 | 0.57 | 0.57 | 1.46 | 1.16 | 0.92 | 3.51 | 2.04 |
| 0.31 | 1.44 | 1.41 | 1.19 | 1.16 | 1.14 | 1.14 | 2.91 | 2.32 | 1.84 | 7.02 | 4.08 |
| 0.63 | 2.87 | 2.81 | 2.38 | 2.32 | 2.28 | 2.27 | 5.82 | 4.64 | 3.69 | 14.05 | 8.16 |
| 1.25 | 5.74 | 5.63 | 4.75 | 4.64 | 4.56 | 4.55 | 11.64 | 9.27 | 7.37 | 28.10 | 16.31 |
| 2.50 | 11.5 | 11.3 | 9.5 | 9.3 | 9.1 | 9.1 | 23.3 | 18.5 | 14.7 | 56.2 | 32.6 |
| 5.00 | 23.0 | 22.5 | 19.0 | 18.6 | 18.2 | 18.2 | 46.6 | 37.1 | 29.5 | 112.4 | 65.2 |
| 10.0 | 45.9 | 45.0 | 38.0 | 37.1 | 36.4 | 36.4 | 93.2 | 74.2 | 59.0 | 224.8 | 130.5 |

SYBR-gold nucleic acid binding dye stock, a 10,000× concentrate, is supplied by Invitrogen (Carlsbad, Calif.) and stored at −20° C. The concentrate is allowed to equilibrate to room temperature before diluting 1 to 100 in Hyclone nuclease free water. This is then diluted 1 to 10 in the experimental plate for a final concentrate of 10× for the assay. This is the optimal dilution to achieve linear binding to siRNA duplex at a concentration range of up to 50 μg/mL concentration. For higher concentrations, the amount of dye to use may be further optimized. The values used to generate the standard curve demonstrating linear binding of SYBR-gold to G1498 siRNA is shown in Table 4.

TABLE 4
G1498 siRNA Standard Curve Values

| [G1498] | Mean | |
|---|---|---|
| μg/mL | Fluorescence | Std Dev |
| 0 | 0 | 3.86 |
| 1.56 | 376 | 10.0 |
| 3.13 | 840 | 44.8 |
| 6.25 | 3254 | 91.4 |
| 12.5 | 10591 | 762 |
| 25.0 | 26276 | 1497 |
| 50.0 | 36543 | 240 |

Samples are mixed directly in the 384 well analysis plate. First 5 μL SYBR-gold dye is pipetted into each well with a multichannel pipette, touching the tip to the bottom of the well to draw out the solution completely. Then 22.5 μL of 2× peptide solution is added with a single channel pipette. Since SYBR-gold will not bind to peptide (and peptide samples without siRNA will verify this assumption), there will be no bleaching effect on fluorescence. Finally, 22.5 μL of 2× siRNA is added with a multichannel pipette. The plate is covered immediately with foil and tapped gently to mix and draw down any droplets on the side of the well.

Fluorescence is measured using the SpectraMax fluorescent plate reader from Molecular Devices (Sunnyvale, Calif.). Plate settings include shaking before reading, one read per well, with excitation wavelength of 495 nm and emission wavelength of 537 nm. The plate is read within 30 minutes of the addition of the siRNA.

### Scatchard Plot

Scatchard Plot is a plot of peptide binding ([peptide]bound/[peptide]free) vs. [peptide]bound. The slope of the linear regression of this plot is −1/Kd and Bₘₐₓ is the y-intercept. Since the concentration of free and bound peptide cannot be measured directly, indirect measurements of siRNA is used for the calculation. Free siRNA is determined from measured fluorescence using the standard curve. Bound siRNA is determined from the standard curve by mass balance from the known initial siRNA concentration (10 μg/mL). Bound peptide is calculated from bound siRNA by assuming the (siRNA:Peptide) bound molar ratio is equal to the (siRNA:Peptide) charge ratio for a single molecule pairing. From this calculated bound peptide amount, the free peptide is calculated by mass balance.

### Example 2

### Peptide Synthesis and Polymerization

The following test peptides with the general amino acid sequence found in Table 6 were synthesized, purified and characterized.

Peptides were synthesized by solid phase peptide synthesis using the multiple peptide synthesizer (Advanced Chem Tech APEX 396). Briefly, all peptides were synthesized from C-terminal to N-terminal by using a standard Fmoc strategy with HOBt and DIC double coupling followed by N-capping with Acetic anhydride and DIPEA to avoid deletion sequences. The trityl group was used to protect the side chains of Cys and His while the Lys and Trp side chains were protected with tert-butoxycarbonyl. For solid support, we used Wang resin with first amino acid loaded (0.45 mmol/μm) and synthesis was start with 90 umols scale.

After completion of synthesis, these peptides were cleaved from the resin, and side-chain protecting groups were simultaneously removed by reaction with TFA/EDT/water (95:2.5:2.5 v/v/v) for 1 and ½ hrs at RT. Then, cleaved peptides were removed from the resin and precipitated with cold ether. The precipitate was separated from liquid and freeze dried. The solid was dissolved in 0.1% TFA and the yield of crude peptide was measured on the basis of Trp absorbance at 280 nm (ε₂₈₀ nm=5600 cm⁻¹ mol⁻¹). Peptide purity was determined by analytical HPLC prior to purification by preparative HPLC. Analytical RP-HPLC was perform by injecting 5 nmol onto a Vydac C₁₈ analytical column (0.47×25 cm) eluted with a gradient 5-15% of acetonitrile/30 min with 0.1% TFA in water at 1 ml/min while monitoring the UV absorbance at 280 nm. Peptides were then purified to homogeneity using RP-HPLC by injecting up to 4 umol onto a Vydac C18 semipreparative column (2×25 cm) eluted with gradient 5-15% of ACN with 0.1% TFA in water at 10 mL/min while monitoring the UV absorbance at 280 nm. The major peak eluting near 20 min was pooled from multiple runs, concentrated by rotary evaporation, lyophilized, and stored dry at −20° C. Purified peptides were reconstituted in 0.1% TFA for calculation of the yield on the basis of Trp absorbance at 280 nm. Results are shown in Table 5.

TABLE 5
Determination of Purified Peptide Yield

| | Starting | | | |
|---|---|---|---|---|
| | Scale of | Crude Peptide | Purified Peptide | Purified Polymerized |
| | Synthesis | Amount (umol) & | Amount (umol) & | Peptide Amount |
| Peptide No. | (umol) | Yield (%) | Yield (%) | (umol) & Yield (%) |
| NT-18 | 90 | 55.45, 61.6% | 26.69, 29.6% | 14.42, 16.0 |
| NT-19 | 90 | 47.88, 53.2% | 22.9, 25.4% | 17.94, 19.93 |
| NT-20 | 90 | 48.77, 54.1% | 20.94, 23.2% | 18.25, 20.2 |
| NT-21 | 90 | 46.09, 51% | 17.04, 18.9% | 14.91, 16.56 |
| NT-22 | 90 | 46.09, 51% | 18.1, 20.1% | 10.55, 11.7 |
| NT-23 | 90 | 47.85, 53.1% | 19.0, 21.1% | 16.05, 17.8 |
| NT-24 | 90 | 35.95, 39.8% | 15.8, 17.5% | 13.47, 14.9 |
| NT-25 | 90 | 40.7, 45.2% | 12.46, 13.8% | 9.91, 11.0 |

Purified peptides were characterized by LC-MS by injecting 2 nmol onto a Vydac C18 reversed-phase HPLC column (0.47×25 cm) eluted at 0.7 mL/min with 0.1% TFA and an acetonitrile gradient of 5 to 15% over 30 min.

Once purified, each peptide may be polymerized at 10 mM pH 8.0 to form the following polymers of estimated molecular weight (MW) shown below in Table 6. The average molecular weight of the polymers is determined using a calibrated GPC-HPLC and SDS-PAGE. The current estimated MW for the peptides ranges from 80-100 kDa. This technique is known as “pre-polymerization” and allows for the generation of suitably large peptides that bind and condense siRNA sufficiently for in vivo efficacy. An alternative approach to generate condensed nanoparticles is to condense the siRNA with the non-polymerized peptides and then initiate polymerization via pH adjustment. This is known as template polymerization. Both approaches are explored to determine the physical properties of the particles and their impact on efficacy.

The peptides are kept as the trifluroacytate (TFA) salt form following synthesis. The number of peptide sequences and the precise sequences is guided by the result of formulation studies described herein. In general, peptides have two terminal cysteine (Cys) residues, but may also include more to increase siRNA stability. The Cys residues are added to enable reversible cross-linking via disulfide bonding of the peptides to create polymerization of the peptides. The peptides have one or more histidine (His) residues, preferably two, to promote endosomal escape. A total of two histidines per peptide represents an optimal level. The peptides also contain a tryptophan (Trp) residue to facilitate quantification of the peptides.

Peptides were polymerized using 70% 150 mM Sodium Chloride, 10 mM Sodium phosphate buffer pH 7.4 and 30% DMSO at room temperature for 24 hours. To confirm the polymerization, we characterized all peptides using analytical RP-HPLC, injecting 5 nmol onto a Vydac C₁₈ analytical column (0.47×25 cm) eluted with a gradient 5-50% of acetonitrile/30 min with 0.1% TFA in water at 1 ml/min while monitoring the UV absorbance at 280 nm. In these experiments, it was noted that each polymeric peptide elutes as a broad peak approximately 5 minutes later than any residual amount (about 5%) of monomeric pepetide.

Polymerized peptides were purified on Sephadex G-50 column (2×120 cm) with eluent 0.1% TFA. Fractions containing polymeric peptides were collected, pooled and freeze dried. Purified peptide polymers were then characterized by analytical RP-HPLC by injecting 5 nmol onto a Vydac C₁₈ analytical column (0.47×25 cm) eluted with a gradient 5-50% of acetonitrile/30 min with 0.1% TFA in water at 1 ml/min while monitoring the UV absorbance at 280 nm. Purified peptide polymers were also characterized by LC system, by injecting 5 nmol onto a Vydac C18 reversed-phase HPLC column (0.47×25 cm) eluted at 0.7 mL/min with 0.1% TFA and an acetonitrile gradient of 5 to 50% over 30 min.

In further analysis, each peptide was also analyzed using an Agilent HPLC method before and after reduction with TCEP. In this regard, purified peptide polymers were reduced by TCEP (peptide polymer:TCEP::1:20 mol ratio) to confirm that they are reversibly capable of returning to a monomer form and characterized by LC-MS by injecting 5 nmol onto a Vydac C18 reverse-phase HPLC column (0.47×25 cm) eluted at 0.7 mL/min with 0.1% TFA and an acetonitrile gradient of 5 to 50% over 30 minutes. Additional characterization was performed by conventional SDS-PAGE.

The MW of purified peptide polymers were characterized by GPC HPLC by injecting 30 ug onto a Shodex OH Pak SB-800 HQ column (7.8×300 mm) eluted by 0.5 M sodium chloride and 50 mM sodium phosphate (pH 7.0) with 10% methanol at a flow rate 0.7 ml/min while monitoring by refractive index detector. To compare the GPC-HPLC data of peptides polymer, we used polylysine standards by injecting 30 ug at same conditions as mentioned above.

In these examples, the amino acid sequence of the peptide is represented as follows: NH₂—(X)_{N}-amide, where N is five to about 20 and X is an positively charged amino acid (e.g., Lysine or Arginine).

Within Table 6, the abbreviation “Ac” means the peptide is acetylated, “PEG2K’ means the peptide is conjugated with poly(ethylene glycol) (PEG) with a molecular weight of 2 kiloDaltons (kD), “PEG10K” means the peptide is conjugated with a 10 kD PEG and “Mal” means maleimido.

TABLE 6

Synthesized Peptides

Estimated
MW
(Daltons)

| | | |
|---|---|---|
| Name of Peptide | mon- | poly- |
| Amino Acid Sequence | omer | mer |

| | | | |
|---|---|---|---|
| NT-18 | Cys-Trp-(Lys)₄-Cys | 922 | 11707 |
| | (SEQ ID NO: 2) | | |
| NT-19 | Cys-Trp-(Lys)₆-Cys | 1178 | 3390 |
| | (SEQ ID NO: 3) | | |
| NT-20 | Cys-Trp-(Lys)₈-Cys | 1435 | 5936 |
| | (SEQ ID NO: 4) | | |
| NT-21 | Cys-Trp-(Lys)₁₀-Cys | 1691 | 42011 |
| | (SEQ ID NO: 5) | | |
| NT-22 | Cys-Trp-(Lys)₄-Cys-(Lys)₄-Cys | 1538 | 6571 |
| | (SEQ ID NO: 6) | | |
| NT-23 | Cys-Trp-(Lys)₄-His-(Lys)₄-Cys | 1572 | 12520 |
| | (SEQ ID NO: 7) | | |
| NT-24 | Cys-Trp-His-(Lys)₄-His-(Lys)₄- | 1709 | 740 |
| | Cys | | |
| | (SEQ ID NO: 8) | | |
| NT-25 | Cys-Trp-His-(Lys)₄-His-(Lys)₄- | 1846 | 502 |
| | His-Cys | | |
| | (SEQ ID NO: 9) | | |
| PN861-2 | Ac-(Arg)₉-amide | | |
| | (SEQ ID NO: 10) | | |
| PN859-2 | Ac-(Arg)₁₈-amide | | |
| | (SEQ ID NO: 11) | | |
| PN860-2 | Ac-(Lys)₉-amide | | |
| | (SEQ ID NO: 12) | | |
| PN858-1 | Ac-(Lys)₁₈-amide | | |
| | (SEQ ID NO: 13) | | |
| PN924-1 | NH₂-(Lys)₂₀-amide | | |
| | (SEQ ID NO: 14) | | |
| PN923-1 | NH₂-(Lys)₂₅-amide | | |
| | (SEQ ID NO: 15) | | |
| PN0933-2 | PEG2k-Mal-(Lys)₁₀-amide | | |
| | (SEQ ID NO: 16) | | |
| PN0930-2 | PEG2k-Mal-(Lys)₁₅-amide | | |
| | (SEQ ID NO: 17) | | |
| PN0925-2 | PEG2k-Mal-(Lys)₂₅-amide | | |
| | (SEQ ID NO: 18) | | |
| PN0931-2 | PEG10k-Mal-(Lys)₁₅-amide | | |
| | (SEQ ID NO: 19) | | |
| PN907-2 | PEG10k-Mal-(Lys)₃₀-amide | | |
| | (SEQ ID NO: 20) | | |
| PN0602-2 | Ac-KGSKKAVTKAQKKDGKKRKRS | | |
| | RKESYSVYVYKVLKQ-amide | | |
| | (SEQ ID NO: 21) | | |
| PN0593 | NH₂-VRLPPPVRLPPPVRLPPP-amide | | |
| | (SEQ ID NO: 22) | | |
| PN0594 | NH2-PLKPLKPLKPLKPLKPLK-amide | | |
| | (SEQ ID NO: 23) | | |

### Example 3

### Degree of Peptide/siRNA Condensation at Varying N:P Ratios

The relative binding of various peptides to siRNA via a rapid screen is assessed by indirect measurement of the displacement of SYBR-gold nucleic acid binding dye. A buffered mixture of siRNA, peptide and SYBR-gold is prepared in the measurement plate in duplicate such that the peptide and SYBR-gold dye undergoes simultaneous competitive binding of the siRNA. The concentration of siRNA is fixed at 10 μg/mL and is combined with a titration of each peptide ranging in a concentration that corresponds to a peptide:siRNA charge ratio between 0.05 to 10. Since SYBR-gold dye only fluoresces when bound to siRNA, peptide binding to the siRNA is inhibits binding of the dye and consequently reduces the fluorescence. Therefore, the amount of fluorescence correlates inversely to the binding of the peptide to the siRNA.

The data is summarized below in Table 7. The values are compared to the positive control peptide PN602-2 (36-mer H₂B peptide) that exhibited a Kd of 0.640 and a Bₘₐₓ of 0.270. A greater Kd value indicates greater binding affinity between the peptide and the siRNA (i.e., a greater degree of condensation).

TABLE 7
Summary Peptide/siRNA Condensation

| | Binding Assay | | Scatchard Analysis | |
|---|---|---|---|---|
| | Max Bound | Corresponding | | Bₘₐₓ |
| | siRNA | charge ratio | Kd | (μMbound/ |
| Peptide Number | (mole %) | (N/P) | (μM⁻¹) | μMfree) |
| PN0907-2 | 58% | 0.63 | NA | NA |
| PN0859-2 | 64% | 0.63 | 0.929 | 0.495 |
| PN0861-2 | 68% | 1.25 | 0.771 | 0.198 |
| PN0602-2 | 73% | 2.5 | 0.640 | 0.270 |
| PN0924-1 | 71% | 1.25 | 0.426 | 0.883 |
| PN0858-1 | 66% | 1.25 | 0.348 | 0.828 |
| PN0933-2 | 39% | 1.25 | 0.326 | 0.241 |
| PN0930-2 | 48% | 1.25 | 0.250 | 0.560 |
| PN0860-2 | 43% | 0.63 | 0.204 | 0.379 |
| PN0925-2 | 59% | 1.25 | 0.150 | 2.077 |
| PN0931-2 | 47% | 1.25 | 0.130 | 0.992 |
| PN0923-1 | 71% | 1.25 | 0.118 | 3.071 |

The results in Table 7 show that poly-arginines (PN861-2 and PN859-2) bind to siRNA with the greatest affinity. The control peptide PN602-2 was the next strongest binder next to the poly-arginine peptides. Further, PEGylation, in general, tends to the decrease apparent binding constant. Finally, the data from this experiment indicates that poly-lysines greater than length scale of siRNA (i.e., 20 charges) decreased ability to bind siRNA relative to the other cationic polymer peptides.

In addition, each of the polymeric peptides NT-18 through NT-25 was evaluated for their ability to condense siRNA. Each polymeric peptide was titrated with siRNA containing thiazole orange. The fluorescence intensity provides a measure of the equivalence point resulting from complete condensation. siRNA (1 μg based on absorbance) was combined with varying amount of peptide polymer and 0.1 g thiazole orange in 1 ml with Hepes buffer mannitol (5 mM HEPES, 5% mannitol, pH 7.5). The Fluorescence used Ex: 498 nm and Em: 546 nm. In this experiment, each peptide was shown to condense siRNA.

### Example 4

### Metabolic Stability of Peptide/siRNA Condensates

The present Example presents a method for determining the metabolic stability of peptide/siRNA condensates at varying N:P ratios. The peptide/siRNA condensates are challenged with RNAse and are analyzed by agarose gel electrophoresis analysis.

A 20 μg aliquot of siRNA duplex alone or with a peptide is mixed with 200 μl of fresh rat plasma incubated at 37° C. At various time points (0, 30, 60 and 20 minutes), 50 μl of the mixture are taken out and immediately extracted by phenol:chloroform. SiRNAs are dried following precipitation by adding 2.5 volumes of isopropanol alcohol and subsequent washing step with 70% ethanol. After dissolving in water and gel loading buffer the samples are analyzed on 20% polyacrylamide gel, containing 7 M urea and visualized by ethidium bromide staining and quantitated by densitometry. The level of degradation at each time point may be assessed by electrophoresis on a PAGE gel.

### Example 5

### Stabilization of Peptide/siRNA Condensates by Cross-Linking

The peptide/siRNA condensates may be cross-linked with varying amounts of glutaraldehyde or by the formation of Cys mediated disulfide crosslinks. This is done to further stabilize the peptide:siRNA condensates and allow for modulation of surface zeta potential, which plays an important role in the non-specific recognition and opsonization of the particles during in vivo systemic circulation.

In a separate experiment, glutathione (GSH) was added to disulfide cross-linked peptide siRNA particles (which may be referred to as nanoparticles) in order to detect siRNA release from disulfide cross-linked peptide siRNA condensates. The reduction of the peptide polymers, leading to dissociation and release of siRNA was monitored by continues measurement of the fluorescence intensity of glutathione over 15 minutes. Briefly, siRNA nanoparticles (N/P: 5 ug siRNA/4 nmols peptide polymer) were treated with reducing agent glutathione (GSH, 0.5 mg scale) and thiazole orange (0.1 ug) in 1 ml sample in HBM buffer pH 7.5. The fluorescence intensity was monitored over 15 min at Ex 498 nm and Emi 546 nm. Samples tested were (1) siRNA and Thiazole orange (No peptide polymer, No GSH), (2) Peptide polymer, siRNA and Thiazole orange (No GSH), and (3) Peptide polymer, siRNA, Thiazole orange and GSH. Samples were prepared as shown in Table 8.

TABLE 8
Sample Preparation For Determination of GSH Mediated siRNA Release

| Sample | NT-Peptide | | | | | Total |
|---|---|---|---|---|---|---|
| No. | Polymer | HBM | siRNA | Thiazole Orange | GSH | Volume |
| 1 | NO | 930 μl | 5 ug/50 μl | 0.1 ug/20 μl | No | 1 ml |
| 2 | 4 nmol/10 μl | 920 μl | 5 ug/50 μl | 0.1 ug/20 μl | No | 1 ml |
| 3 | 4 nmol/10 μl | 915 μl | 5 ug/50 μl | 0.1 ug/20 μl | 0.5 mg/5 μl | 1 ml |

The data from this experiment show that the fluorescence intensity increases to a maximum within 100 seconds and then becomes constant. We interpret this to be complete release of siRNA even thought the fluorescence intensity is low compared to control 1. We attribute this lower intensity to some quenching by glutathione. We have compared the intensity with increasing amounts of glutathione and found that quenching does occur.

We observe that most of the polymers can be reduced, leading to increases quenching. The exception seems to be NT-21 which contains the longest poly-Lys of 10. This could indicate that a monomeric peptide of this length continues to bind to siRNA even when the polymer is reduced.

### Example 6

### Particle Size, Condensation of siRNA with PEG Modified Peptide

In this experiment, we determined the ability to form PEG-Peptide-siRNA condensates, and demonstrate that high-concentrations siRNA condensates could be formed while maintaining particle size, and demonstrate the ability to extrude siRNA PEG-peptide-siRNA particles to smaller particles, and demonstrate the ability to freeze dry and reconstituted PEG-peptide-siRNA particles showing that particle size is maintained.

As exemplified in this experiment the peptide contained a maleimide linkage (designated PEG-Mal-CWK₁₈). It is appreciated that a reducible S—S bond located between the PEG moiety and the siRNA may also be used. In either case, the PEG used is 5000 Daltons in size.

Briefly, siRNA (1 μg) was combined with varying amount of peptide and 0.1 ug thiazoleorange in 1 ml with Hepes buffer mannitol (5 mM HEPES, 5% mannitol, pH 7.5). The fluorescence was measured with an Ex: 498 nm and Em: 546 nm. This experiment established that a PEG-modified peptide could condense siRNA at ratio of approximately 0.3 nmol peptide per μg of siRNA.

In addition, we evaluated the particle size of PEG-peptide-siRNA condensates measured as a function of concentration. In this evaluation, the concentration of siRNA nanoparticles was varied from 50 to 2000 μg/ml at a constant ratio of PEG-peptide of 0.3 nmol per μg of siRNA. siRNA nanoparticles were prepared at a desired concentration of 50 μg/ml to 2000 μg/ml then diluted to 1 ml in Hepes buffer mannitol (5 mM HEPES, 5% mannitol, pH 7.5) (Filter) to form a siRNA nanoparticle of 50 μg/ml just prior to performing particle size analysis. The results indicate that PEG-peptide siRNA condensates can be prepared at concentration range from about 50 to about 2000 μg/ml without the formation of large particles. Such data indicates that relatively small volumes containing significant amounts of PEG-peptide-siRNA condensates may be used to administer therapeutic levels of siRNA to a subject patient in need of such treatment. The results demonstrate that the particle size of PEG-peptide-siRNA particles remains constant following freeze drying and reconstitution.

In another study, we evaluated the particle size after reconstitution of freeze dried PEG-peptide-siRNA condensates. Such data may impact, for example, drug packaging, shipping and stability. The results show that the particle size of PEG-peptide-siRNA particles remains constant following freeze drying and reconstitution, before and after freeze drying particle size was in a range of about 130-135 nm. Further studies using high pressure extrusion of PEG-peptide-siRNA condensates through for example 50 or 30 nm membranes may be used in order to provide particle sizes in a range from about 30 to 50 nm.

### Example 7

### Particle Size, Zeta Potential and Stability as a Function of the Degree of Peptide/siRNA Cross-Linking

The particle size, zeta potential and stability of the peptide/siRNA condensate are determined as a function of the degree of cross-linking. Zeta potential measures the surface charge present on peptide/siRNA condensates.

The peptide/siRNA condensates are analyzed for surface charge in order to select those condensates that maintain a zeta potential of approximately +10 millivolts (mV) or less. Peptide/siRNA condensates are limited to a cationic charge of +10 mV or less in order to prevent promiscius and non-specific binding of the condensate while in a biological environment. Cross-linking the peptide/siRNA condensates with gluteraldehyde reduces the free-unbound cationic charges, thus reducing promiscuous non-specific binding.

Formulations for particle size analysis were prepared in Hepes 11 mM pH7.4, at a siRNA concentration of 0.8 uM (11.59 μg/ml) and then diluted into OPTI-MEM. For nanoparticle size analysis of siRNA/peptide polymers, we used 1 ug RNA/0.8 nmols peptide polymer (based on monomer). The total vol of sample was 1 ml containing 50 ug RNA and 40 nmol peptide polymer in Hepes buffer mannitol (5 mM HEPES, 5% mannitol, pH 7.5) (Filter). The particle size analysis was performed on a Zeta Plus/Zeta potential analyzer.

Briefly, buffer was added first and then concentrated siRNA was added followed by votexing for 3 seconds (at full speed). The peptide polymer was then added followed by votexing for an additional 6 seconds (at full speed). Formulations were maintained at room temperature for 10 minutes. In a 96-well plate format, formulations were then diluted 20× for a final siRNA concentration of 10 nM (6.25 μl into 118.75 μl of OPTI-MEM) or 2× for a final concentration of 100 nM (62.5 μl of formulation into 62.5 μl OPTI-MEM). After dilution, formulations were mixed gently. For positive control preparation, 0.5 μl of HiPerFect was diluted into 12.5 μl of OPTI-MEM and 0.5 μl of siRNA into 12.5 μl of OPTI-MEM, respectively; and incubated at room temperature for 5 minutes, and then combined diluted siRNA and diluted HiPerFect, and incubated at room temperature for 15 minutes. A formulation pH of about 7.4 was chosen in order to minimize reduction of peptide that is polymerized through cycteine disulfide bonds.

Particle size was measured 24 hours after formulation of siRNA 0.8 μM/peptide polymers (N/P=2). This particle size analysis showed that NT-18 formed a particle size of about 105 nm, NT-19 formed a particle of about 78.7 nm in size, NT-20 a particle of about 67.6 nm, NT-21 a particle of about 55.7 nm in size, NT-22 a particle of about 90.8 nm in size, NT-23 a particle size of about 65.7 nm, NT-24 a particle size of about 82.2 nm, and NT-25 a particle of about 64.6 nm in size. In summary, all formulation tested in this study formed particles at about or less than about 100 nm.

In a subsequent study, particle size was shown to increase in a time-dependent manner over a period of 4.5 hours. For example, at T=0 the particle size was about 80 nm, at T=50 minutes about 500 nm, and at T=200 minutes the particle size was about 700 nm. It is possible that this observation represents aggregation of peptide/siRNA condensates.

### Example 8

### Cell-Uptake and Gene Targeted Knockdown Efficacy of Various Peptide/siRNA Formulations

This example discloses suitable methodology for determining whether universal-binding nucleotide comprising siRNA of the present disclosure are capable of enhancing the ability of the siRNA to downregulate expression of one or more target genes. Example cells lines used to test peptide/siRNA condensate include 9L/LacZ and mouse tail fibroblast (MTF) cells.

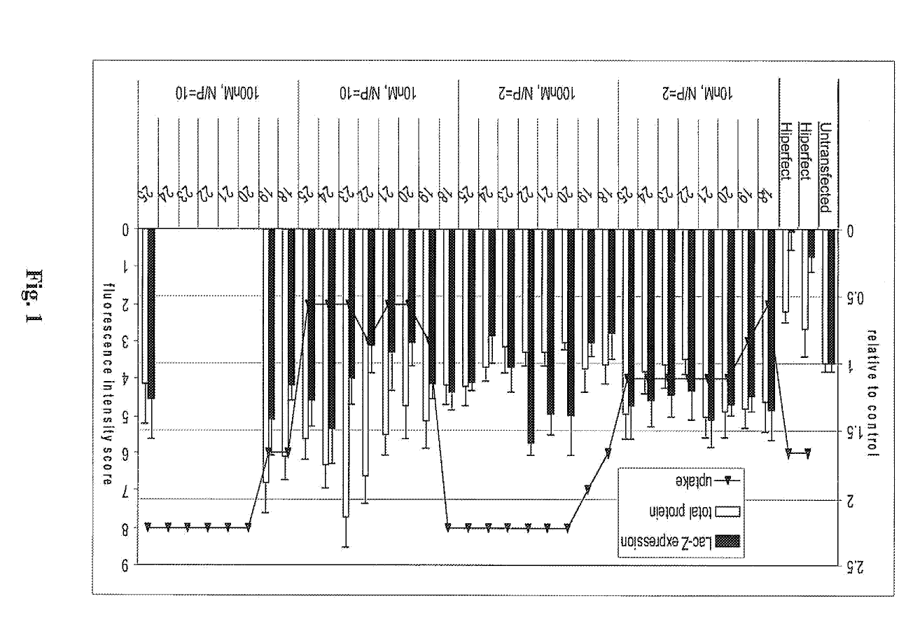

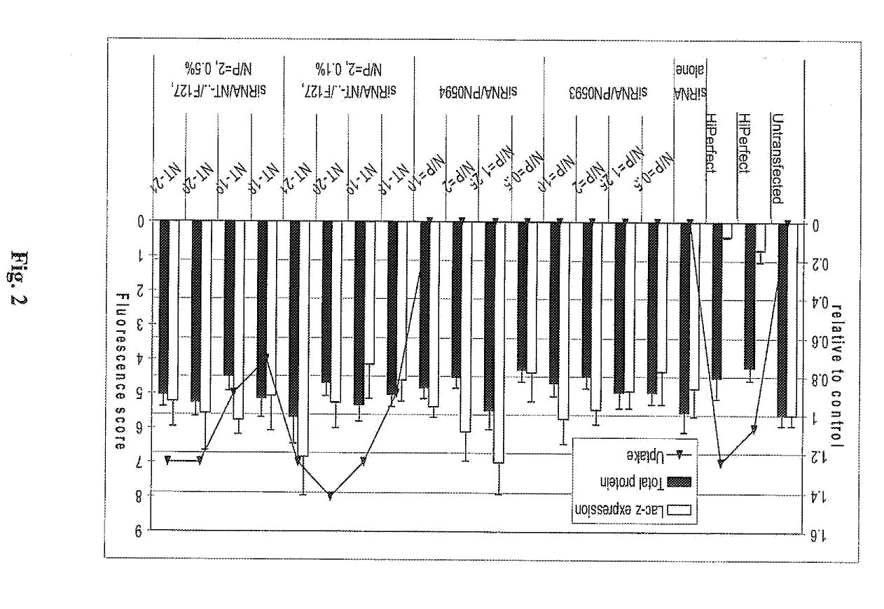

SiRNA (SEQ ID NO:1) cell-uptake and knockdown activity are determined by transfecting cells with the peptide/siRNA condensates. A random siRNA sequence may be used as a negative control. The data accumulated from two such in vitro cell-based studies (study 1 and study 2) are shown in FIGS. 1 and 2, respectively. The cell line designated 9L/LacZ was used for transfection of siRNA/peptide condensates in both study 1 and study 2.

In a first study (FIG. 1), siRNA/peptide condensate formulations comprising the peptides NT-18 through NT-25 were evaluated for cellular uptake and knockdown of target gene expression using the cell line designated 9L/LacZ. In a second study, siRNA/peptide condensate formulations comprising the peptides PN0593 or PN0594 were evaluated, along with siRNA/peptide condensate formulations comprising pluronic F127 (to reduce aggregation) and the peptides NT-18, NT-19, NT-20 and NT-21.

For study 1 and 2, 7,000-10,000 9L/LacZ cells (passage 5-25) were plated in cell culture media in each well of a 96-well plate and grown overnight. Cells (in 75 μl of media) were transfected by adding 25 μl of each formulation to be tested; 4 hours thereafter 100 μl of fresh media is added to each well, which media is replaced with 100 μl of fresh media approximately 16 hours later. Positive control transfections with HiPerFect were performed according to manufacturer recommendations (Qiagen, Inc., Valencia, Calif.). Cells were harvested 3 days after transfection by the addition of 50 μl of lysis buffer for β-galactosidase and Micro BCA assays.

For study 1 and 2, a Micro BCA assay (Pierce Chemical Company, Rockford, Ill.) was performed in order to measure total protein concentration, as follows: 9L/LacZ cells were washed 1× with phosphate buffered saline (PBS). Then, 50 μl of M-PER® Reagent was added to each well and incubated for 15 minutes at room temperature. After incubation, 10 μl of each lysate was transferred to a new well (using a 96-well plate format) containing 140 μl of dH₂0, and then 150 μl of Micro BCA working solution was added. The plates were covered, incubated at 37° C. for 2 hours and then the absorbance measured at 562 nm was recorded. For study 1 and 2, a β-galatosidase assay was performed in order to measure siRNA modulation of target gene expression. Briefly, cells were washed 1× with PBS and then 50 μl of M-PER® Reagent was added to each well and incubated at room temperature for 15 minutes. Thereafter, 30 μl of lysate was transferred to a new plate along with the addition of 30 μl of All-in-One™ β-galatosidase Assay Reagent; the plates were covered, incubated for 30 minutes at 37° C., after which absorbance is measured at 405 nm.

The results of this study 1 (FIG. 1) show that uptake of peptide/siRNA condensates was most pronounced for compositions formulated at 100 nM with an N/P=2, and 100 nM with an N/P=10. As used herein, N/P means nitrogen to phosphate charge ratio where nitrogen reflects peptide charge and phosphate reflects siRNA charge. Uptake of compositions formulated with peptide/siRNA condensates at 10 nM and an N/P=2 or N/P=10 were lower than that observed with formulations transfected at a concentration of 100 nM. In this experiment, formulations comprising peptides designated NT-18, NT-19 and NT-24 show a reduction of target gene (i.e., Lac-Z) expression over a range from about 15% to about 25% (each at 100 nM, N/P=2), with no apparent toxicity as total protein expression was essentially unchanged from control values. Positive control formulations (i.e., formulations transfected along with HiPerFect) showed a larger reduction in target gene expression, with a significant increase in toxicity. All values presented are relative to negative (i.e., non-transfected) control samples.

In a second study (FIG. 2), formulations comprising siRNA/PN0593 and siRNA/PN0594 condensates were evaluated (at varying N/P ratios) along with formulations comprising siRNA/NT-18 condensates, siRNA/NT-19 condensates, siRNA/NT-20 condensates and siRNA/NT-21 condensates (each at a concentration of 100 nM and an N/P=2). In this experiment, the positive control comprised HiPerFect and the negative control was un-transfected cells. A siRNA alone control was also included in this experiment. The results show that uptake of siRNA/PN0593 or siRNA/PN0594 was very low. Formulations comprising pluronic F127 and siRNA/NT-18, siRNA/NT-19, siRNA/NT-20 or siRNA/NT-21 condensates showed good uptake, and easily detectable knockdown of gene expression ranging from about 20% (NT-18) to about 27% (NT-19); both of these formulations were evaluated at concentrations of 100 nM, an N/P=2, and contained 0.1% pluronic F127.

Collectively, these data indicate that, delivery of siRNA/peptide condensates comprising peptides such as NT-18 or NT-19 (in the presence or absence of pluronic F127) may be used for the targeted knockdown of gene expression following uptake into the appropriate cell.

Although the foregoing disclosure has been described in detail by way of example for purposes of clarity of understanding, it will be apparent to the artisan that certain changes and modifications may be practiced within the scope of the appended claims which are presented by way of illustration not limitation. In this context, various publications and other references have been cited within the foregoing disclosure for economy of description. Each of these references is incorporated herein by reference in its entirety for all purposes. It is noted, however, that the various publications discussed herein are incorporated solely for their disclosure prior to the filing date of the present application, and the inventors reserve the right to antedate such disclosure by virtue of prior disclosure.

What is claimed is:

1. A composition for delivery of a RNA molecule to a cell, comprising: a double stranded RNA (dsRNA) molecule of about 15 to about 40 base pairs; and a cationic polymer peptide comprising at least two cysteine residues and at least one tryptophan residue with one or more repeats consisting of between about two to about 20 positively charged residues.

2. The composition of claim 1, wherein the peptide comprises two or more cysteine residues and one or more tryptophan residues with one or more repeats consisting of positively charged residues.

3. The composition of claim 1, wherein the peptide comprises two or more cysteine residues, one or more tryptophan residues, two or more histidine residues with one or more repeats consisting of positively charged residues.

4. The composition of claim 1, wherein the cationic polymer peptide has an amino acid sequence comprising Cys-Trp-(Lys)_{X}-Cys (SEQ ID NO:29); Cys-Trp-(Lys)_{X}-Cys-(Lys)_{X}-Cys (SEQ ID NO:30); Cys-Trp-(Lys)_{X}-His-(Lys)_{X}-Cys (SEQ ID NO:31); Cys-Trp-His-(Lys)_{X}-His-(Lys)_{X}-Cys (SEQ ID NO:32) or Cys-Trp-His-(Lys)_{X}-His-(Lys)_{X}-His-Cys (SEQ ID NO:33), wherein X is greater than one and the total number of non-lysine residues is less than 18.

5. The composition of claim 1, wherein the cationic polymer peptide has an amino acid sequence comprising Cys-Trp-(Lys)₄-Cys (SEQ ID NO:2) or Cys-Trp-(Lys)₆-Cys (SEQ ID NO:3).

6. The composition of claim 1, wherein the peptide is pegylated.

7. The composition of claim 1, wherein the peptide is acetylated.

8. The composition of claim 1, wherein the composition is reacted with a dialdehyde to cross-link a multiplicity of peptides.

9. The composition of claim 1, wherein the peptides cross-link after binding to dsRNA by forming interpeptide disulfide bonds.

10. The composition of claim 1, wherein the peptide is derivatizing with a single N-glycan.

11. A method of stabilizing a double stranded ribonucleic acid (dsRNA)-peptide condensate, comprising:
a. preparing a dsRNA-peptide condensate by mixing a dsRNA with a peptide in water, and
b. cross-linking at least a portion of the peptides within the condensate.

12. The method of claim 11, further comprising the steps of:
a. selecting a peptide comprising two or more lysine residues, and
b. cross-linking the peptides after binding to dsRNA by reacting with a dialdehyde.

13. The method of claim 11, further comprising the steps of:
a. selecting a peptide comprising two or more cysteine residues, and
b. cross-linking the peptides after binding to dsRNA by forming interpeptide disulfide bonds.

14. A method for delivering a double stranded (ds)RNA molecule to a cell, comprising:
a. preparing a condensate comprising a dsRNA molecule of about 15 to about 40 base pairs;
b. preparing a dsRNA-peptide condensate by mixing a dsRNA with a peptide in water,
c. cross-linking at least a portion of the peptides within the condensate, and
d. contacting a cell with said condensate.

15. The method of claim 14, further comprising the steps of:
a. selecting a peptide comprising two or more lysine residues, and
b. cross-linking the peptides after binding to dsRNA by reacting with a dialdehyde.

16. The method of claim 14, further comprising the steps of:
a. selecting a peptide comprising two or more cysteine residues, and
b. cross-linking the peptides after binding to dsRNA by forming interpeptide disulfide bonds.

17. A method for inhibiting expression of a gene in a cell comprising
a. preparing a condensate comprising a dsRNA molecule of about 15 to about 40 base pairs;
b. preparing a dsRNA-peptide condensate by mixing a dsRNA with a peptide in water,
c. cross-linking at least a portion of the peptides within the condensate, and
d. treating a cell with said condensate.
